(19) United States
(12) Patent Application Publication
(10) Pub. No.: US 2010/0324454 A1
(43) Pub. Date: Dec. 23, 2010

(54) DECEPTION DETECTION USING OCULOMOTOR MOVEMENTS

(76) Inventors: John C. Kircher, Salt Lake City, UT (US); Anne E. Cook, Salt Lake City, UT (US); Douglas J. Hacker, Salt Lake City, UT (US)

Correspondence Address: Ballard Spahr LLP, SUITE 1000, 999 PEACHTREE STREET, ATLANTA, GA, 30309-3915, US

(21) Appl. No.: 12/820,488

(22) Filed: Jun. 22, 2010

Related U.S. Application Data
(60) Provisional application No. 61/219,194, filed on Jun. 22, 2009.

(51) Int. Cl. A61B 5/103

(52) U.S. Cl. 600/587

(57) ABSTRACT

Embodiments of the present invention relate to a rapid, automated, and objective method for using oculomotor measures to determine whether a person is being truthful or deceitful. More specifically, embodiments of the present invention measure a plurality of oculomotor and behavioral dependent measures while a subject reads and responds to written items that are both related and unrelated to a suspected crime. The oculomotor and behavioral measures may include pupil diameter, response times, the number of fixations during reading, the time spent reading and rereading items, and the rate of eye blinks on the current and subsequent item. Several behavioral and oculomotor measures were diagnostic of deception, and a weighted combination of four of those variables correctly classified 84% of guilty and 89% of innocent subjects.

### CROSS REFERENCE TO RELATED APPLICATIONS

This application claims priority to and benefit of U.S. Provisional Patent Application Ser. No. 61/219,194, filed on Jun. 22, 2009, which is fully incorporated by reference herein and made a part hereof.

### FIELD

The present invention is related to methods and systems for rapid, automated, and objective method for using oculomotor measures to determine whether a person is being truthful or deceitful.

### BACKGROUND

In many instances, including national security, it would be desirable to be able to detect the veracity of a subject; that is, to accurately determine when a subject is being truthful and when they are being deceitful. Government agencies routinely conduct credibility assessments to screen applicants for positions in intelligence, security, law enforcement, immigration, and public transportation. The private sector also has uses for credibility assessment. Errors in classifying a subject as truthful or deceptive in these settings can have serious consequences for the individual and society.

The most common technique to detect deception is a polygraph. A polygraph is an instrument that simultaneously records changes in physiological processes such as heartbeat, blood pressure, respiration and electrical resistance (galvanic skin response or GSR). The polygraph is in credibility assessment by law enforcement (e.g., police departments, the FBI, the CIA), federal and state governments, and numerous private agencies.

The underlying theory of the polygraph is that when a subject is being deceitful they become nervous and as a result cause changes in several physiological processes. A baseline for these physiological characteristics is established by asking the subject questions with known responses. Deviation in physiological processes from the baseline is assumed to correlate with deception.

Despite its widespread use, questions have been raised about the validity of the polygraph for screening and its susceptibility to countermeasures. For example, the National Research Council (NRC) was critical of the polygraph for preemployment screening and highlighted the need for “an expanded research effort directed at methods for detecting and deterring major security threats, including efforts to improve techniques for security screening . . . ” Similarly, self-report integrity tests for screening potential employees have been criticized due to questions about their effectiveness, and behavioral and content analyses have their own shortcomings. One group, for example, reported unacceptable misclassification rates of deceptive subjects (i.e., false negatives) as high as 60% and as high as 37% for truthful subjects (i.e., false positives). Moreover, the polygraph has other requirements that render it impractical as a screening tool in many contexts—it is time consuming, subjective, costly, and requires the attachment of sensors and a trained examiner.

For some time the field has researched the use of other measures in response to behavior changes for detecting deception. Among these, pupil diameter (PD) has been shown to change in response to deception. US Patent Application Publication 2009/0216092 to Waldorf et al. broadly discloses the using pupil diameter as a method for detecting deception although exactly how one skilled in the art would use pupil diameter to discriminate between truthful and deceptive subjects is not taught.

While it has been recognized for decades that changes in pupil diameter correlate with deception, the studies had two major drawbacks. First, pupil diameter was assessed as response to verbal questioning which draws on emotional reactions rather than cognitive responses. Second, pupil diameter as a single measure, while diagnostic in a research setting is generally insufficient on its own for credibility assessment in, for example, preemployment setting. In other words, it has never been shown that pupil diameter alone is sufficient for credibility assessment (i.e., has adequate sensitivity and specificity).

The lack of any viable commercial technology to use pupil dilation and other measures in credibility assessment is supported by the fact that the Department of Homeland Security in December of 2007 released an SBIR entitled “Assess Ability to use Eye Tracking and Pupil Dilation to Determine Intent to Deceive.” SBIR Topic Number: H-SB08.1-001. The request for proposals states that “current but unproven studies suggest that a cognitive decision to deceive or practice deception will result in a increased pupil size due to the greater cognitive processing required in comparison to truthful recall. An assessment study to determine the correlation between pupillometry (dilation and contraction of the pupil relative to observed stimulus or emotion) and intent to deceive is required.”

After recent global events, numerous government agencies have called for immediate development of credibility assessment technologies that can be used to detect a subject's intent to carry out malicious acts. Unanimously, these agencies are calling for a credibility assessment tool that is non-invasive, efficient and rapid. The technology must be flexible and broadly applicable, including the support of employment screening to evaluate the risk of individuals entering transportation and other critical infrastructure (e.g., U.S. borders patrols), as well as a variety of high and low volume venues (e.g., military and civilian checkpoints and airport security checkpoints). Finally, accuracy is of utmost importance. The technology, therefore, should not only be able to accurately predict deceptive subjects (i.e., true positive) and truthful subjects (i.e., true negatives), but also would limit the number of misclassification of truthful (false positives) and deceptive subjects (i.e., false negatives).

### SUMMARY

Embodiments of the present invention provide an improved method of credibility assessment. Unlike previous methods, embodiments of the present invention are well suited for broad applicability at locations such as U.S. borders, military and civilian checkpoints, and airport security checkpoints. Importantly, embodiments of the present invention are noninvasive, highly automated, and does not rely on the subjective opinion of an examiner. Embodiments of the present invention are also diagnostic and applicable to real-world applications that require accurate classification of the veracity of a subject. It is better than previous methods at accurately classifying deceptive subjects (i.e., true positive) and truthful subjects (i.e., true negatives), while limiting the number of misclassification of truthful (false positives) and deceptive subjects (i.e., false negatives). Finally, embodiments of the present invention allow the public or private agency conducting the credibility assessment to adjust the parameters to limit the number of false positives or negatives depending on their desired outcome and practical considerations of the situation (e.g., rapid screening at airports versus employment as a boarder patrol).

Embodiments of the present invention are unlike the polygraph or previous pupil dilation studies where the question were orally presented without placing time constraints on the subject's response. In one embodiment of the present invention, True/False statements are presented to the subject in a written format by a computer and the subject is instructed to respond as quickly and as accurately as possible. Some of the statements refer to the subject's possible deception (e.g., “I falsified information on my pre-employment form.”) and other statements are neutral (e.g., “Today is Tuesday.”). Oculomotor (eye position and pupil size) and behavioral measures (response times and errors) can be collected using well-known methods in the art to analyze the differences between deceptive and innocent subjects.

In another embodiment, independent variables such as motivation and item difficulty are manipulated to determine their effects on oculomotor and behavioral measures of deception. By manipulating independent variables, the investigation demonstrates the conditions under which the proper classification of truthful and deceptive subjects is significantly improved.

The effort exerted by subjects to respond to test items is translated into measurable dependent variables that differ between truthful and deceitful subjects. In one embodiment, data collected may include response time, proportion wrong, number of fixations, first pass duration, reread duration, pupil diameter, item blink rate, and next item blink rate. The presentation of test items, data collection, and analysis can be automated by capturing and processing data from an eye tracker device and a computer. Using the results from the study provided herein, the data can be analyzed to determine the probability that a subject was truthful or deceptive when they read and respond to the statements on the computer-administered test.

Using one of many potential algorithms for selecting and weighing oculomotor and behavioral measures, a weighted combination of four variables that were diagnostic of deception correctly classified 84% of deceptive subjects and 89% of truthful subjects.

Additional advantages will be set forth in part in the description which follows or may be learned by practice. The advantages will be realized and attained by means of the elements and combinations particularly pointed out in the appended claims. It is to be understood that both the foregoing general description and the following detailed description are examples and explanatory only and are not restrictive, as claimed.

### BRIEF DESCRIPTION OF THE DRAWINGS

The accompanying figures, not drawn to scale, which are incorporated in and constitute a part of this specification, illustrate embodiments and together with the description, serve to explain the principles of the methods and systems:

FIG. 1a illustrates an exemplary operating environment in accordance with an embodiment described herein;

FIG. 1b is a block diagram describing some components of a system of one embodiment described herein;

FIG. 2 illustrates a table of means, standard deviations, and ranges for age, grade point average (GPA), self-control, achievement motivation and Marlowe-Crowne;

FIG. 3 illustrates a table of frequencies and percentages for categorical demographic questions;

FIG. 4a illustrates a table of means (and standard deviations) for the dependent variables by motivation, item difficulty, and question type for guilty subjects;

FIG. 4b illustrates a table of means (and standard deviations) for the dependent variables by motivation, item difficulty, and question type for innocent subjects;

FIG. 5 illustrates guilt by question type interaction for response time;

FIG. 6 illustrates guilt by item difficulty interaction for proportion wrong;

FIG. 7 illustrates guilt by question type interaction for number of fixations;

FIG. 8a illustrates guilt by question type interaction for number of fixations for low motivation group;

FIG. 8b illustrates guilt by question type interaction for number of fixations for high motivation group;

FIG. 9 illustrates guilt by question type interaction for first pass duration;

FIG. 10 illustrates guilt by question type interaction for reread duration;

FIG. 11a illustrates guilt by question type by time interaction for PD for guilty subjects;

FIG. 11b illustrates guilt by question type by time interaction for PD for innocent subjects;

FIG. 12 illustrates guilt by question type interaction for next item blink rate;

FIG. 13 illustrates a table of point-biserial correlations (and reliabilities) for easy and mixed item difficulty conditions;

FIG. 14 illustrates a table of point-biserial correlations by repetition for the eight variables selected for possible inclusion in the discriminant function of one embodiment described herein;

FIG. 15 illustrates a table of intercorrelations among RTCrimeNeutral, RTCashExam, NFixCrimeNeutral, NFixCashExam, FirstPassCashExam, RereadCashExam, PDCashExam, and NextItemBlinkCashExam;

FIG. 16 illustrates standardized canonical discriminant function coefficients;

FIG. 17 illustrates a table of functions at group centroids;

FIG. 18 illustrates a table of frequencies (and percentages) of cases correctly classified with the linear discriminant function;

FIG. 19 illustrates a table of frequencies (and percentages) of cases correctly classified with the linear discriminant function using variables from Cook et al. (Cook, A. E., Hacker, D. J., Webb, A., Osher, D., Kristjansson, S., Woltz, D. J., et al., (2008). Lyin' eyes: Oculomotor measures of reading reveal deception), incorporated herein by reference;

FIG. 20 illustrates guilt by achievement motivation interaction for the difference between cash and exam items for RT;

FIG. 22a illustrates the relationship between the sensitivity against 1-specificity for the particular discriminant function; and

FIG. 22b illustrates a receiver operator curve (ROC), which is a plot of the relationship between the sensitivity against 1-specificity for the particular discriminant function.

### DETAILED DESCRIPTION

Embodiments of the present invention may be understood more readily by reference to the following detailed description and the examples included therein and to the figures and their previous and following description.

Before the present systems, articles, devices, and/or methods are disclosed and described, it is to be understood that this invention is not limited to specific systems, specific devices, or to particular methodology, as such may, of course, vary. It is also to be understood that the terminology used herein is for the purpose of describing particular embodiments only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used in the specification and the appended claims, the singular forms “a,” “an” and “the” include plural referents unless the context clearly dictates otherwise. Thus, for example, reference to “a layer” includes two or more such layers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data are provided in a number of different formats and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Embodiments according to the present invention are described below with reference to block diagrams and flowchart illustrations of methods, apparatuses (i.e., systems) according to an embodiment of the invention. Accordingly, blocks of the block diagrams and flowchart illustrations support combinations of means for performing the specified functions and combinations of steps for performing the specified functions.

Embodiments of the present methods and systems may be understood more readily by reference to the following detailed description and the Examples included therein and to the Figures and their previous and following description.

Embodiments of the present invention relates to methods and systems to determining the veracity of a subject. The methods and systems are flexible and intended to be modified to suit the intended purpose of the examiner. In one embodiment, a subject is fitted with an apparatus that is able to detect and, ideally, record a plurality of oculomotor measurements. Once well positioned and determined to be working properly (e.g., calibrated), the subject is presented with a series of test items. In one embodiment, the test items are presented on a computer screen where time to respond and other measures can be recorded. As discussed in greater detail below, the test items are preferably presented in written format and a limit is place on the time the subject has to response to the test item. A combination of independent variables (e.g., sex, age, item difficulty, etc) and dependent measures (e.g., pupil diameter, blink rate, time to response, etc.) are recorded and used to calculate a discriminant function using well-known means in the art to discriminate or classify the subjects of interest. As discussed below, the discriminant function may vary from population to population and use to use. Using appropriate cutoffs for the intended use, the subject is classified as a true positive or true negative. In one embodiment, true positive refers to a deceitful or guilty subject and true negative refers to a truthful or innocent subject. However, one skilled in the art appreciates that these categories are arbitrary and the examiner can vary the lexicon to suit the intended purpose.

Results from a present study described herein indicate that a combination of behavioral and oculomotor measures can be used to detect deception. These results were found in a mock-crime study similar to a forensic situation but also have potential for use in other applications, such as preemployment screening, airport security, law enforcement, etc.

### Exemplary Operating Environment

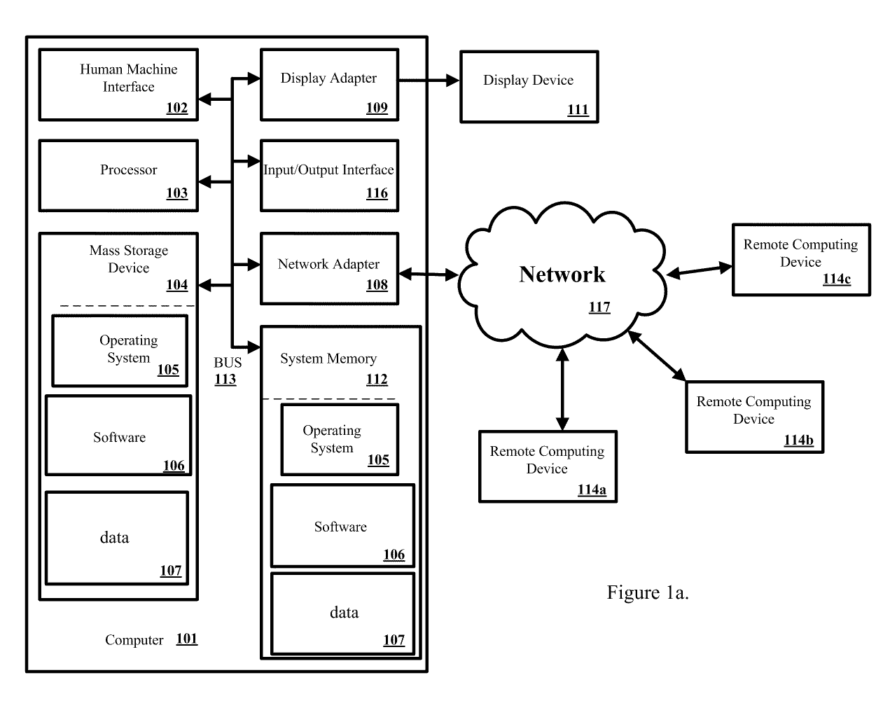

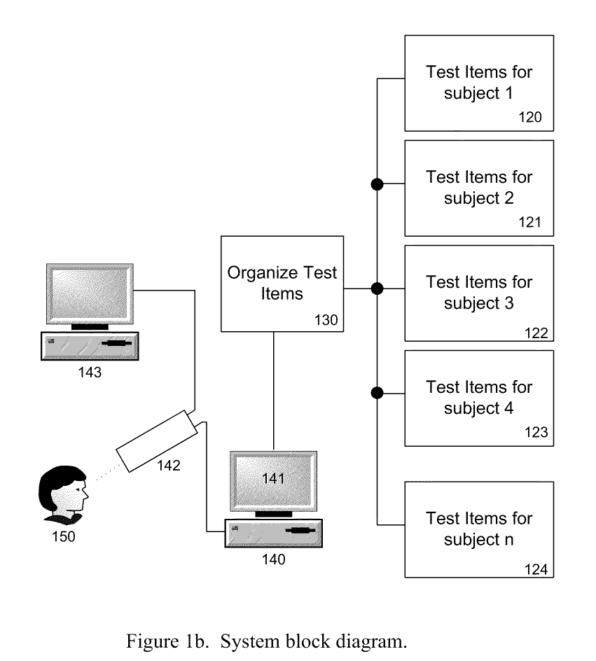

FIG. 1a is a block diagram illustrating an exemplary operating environment for performing the disclosed methods. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

Embodiments of the methods and systems described herein can be operational with numerous other general purpose or special purpose computing system environments or configurations. Examples of well known computing systems, environments, and/or configurations that can be suitable for use with embodiments of the methods and systems described herein comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that embodiments of the systems and methods disclosed herein can be implemented via a general-purpose computing device in the form of a computer 101. The components of the computer 101 can comprise, but are not limited to, one or more processors or processing units 103, a system memory 112, and a system bus 113 that couples various system components including the processor 103 to the system memory 112. In the case of multiple processing units 103, the system can utilize parallel computing.

According to one embodiment, as discussed in more detail below, the processor 103 can be configured to perform one or more of the operations associated with determining the probability that a subject is providing a deceptive answer. For example, according to one embodiment, the processor 103 can be configured to present one or more test items to a subject (e.g., via a display device, discussed below) for the purpose of collecting and analyzing oculomotor data associated with the subject. Alternatively, or in addition, the processor 103 can be configured to collect the oculomotor data including, for example, eye movements, pupil diameter, and/or the like, and/or to analyze the collected data in order to determine the probability that the subject is providing a deceptive response.

The system bus 113 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI) bus also known as a Mezzanine bus. The bus 113, and all buses specified in this description can also be implemented over a wired or wireless network connection and each of the subsystems, including the processor 103, a mass storage device 104, an operating system 105, software 106, data 107, a network adapter 108, system memory 112, an Input/Output Interface 110, a display adapter 109, a display device 111, and a human machine interface 102, can be contained within one or more remote computing devices 114a,b,c at physically separate locations, connected through buses of this form, in effect implementing a fully distributed system.

The computer 101 typically comprises a variety of computer readable media. Exemplary readable media can be any available media that is accessible by the computer 101 and comprises, for example and not meant to be limiting, both volatile and non-volatile media, removable and non-removable media. The system memory 112 comprises computer readable media in the form of volatile memory, such as random access memory (RAM), and/or non-volatile memory, such as read only memory (ROM). The system memory 112 typically contains data 107 and/or program modules such as operating system 105 and software 106 that are immediately accessible to and/or are presently operated on by the processing unit 103.

In another aspect, the computer 101 can also comprise other removable/non-removable, volatile/non-volatile computer storage media. By way of example, FIG. 1a illustrates a mass storage device 104 which can provide non-volatile storage of computer code, computer readable instructions, data structures, program modules, and other data for the computer 101. For example and not meant to be limiting, a mass storage device 104 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 104, including by way of example, an operating system 105 and software 106. Each of the operating system 105 and software 106 (or some combination thereof) can comprise elements of the programming and the software 106.

According to one embodiment, the software 106 can include computer program instructions for instructing the processor 103 to perform one or more of the operations discussed above and below for determining the probability that a subject is providing a deceptive response. For example, the software 106 can include software associated with an eye tracker device (e.g., the Arrington ViewPoint Eye Tracker device (discussed below)); Eyelab 3.0, or similar, software (discussed below) for collecting oculomotor data; and/or software associated with analyzing the collected oculomotor data to determine the probability of a deceptive response.

According to one embodiment, data 107 can also be stored on the mass storage device 104. The data 107 can include, for example, collected oculomotor data associated with a subject (e.g., data corresponding to recorded eye movements, PD, and/or the like). Data 107 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computer 101 via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices can be connected to the processing unit 103 via a human machine interface 102 that is coupled to the system bus 113, but can be connected by other interface and bus structures, such as a parallel port, game port, an IEEE 1394 Port (also known as a Firewire port), a serial port, or a universal serial bus (USB).

In yet another aspect, a display device 111 can also be connected to the system bus 113 via an interface, such as a display adapter 109. It is contemplated that the computer 101 can have more than one display adapter 109 and the computer 101 can have more than one display device 111. For example, a display device can be a monitor, an LCD (Liquid Crystal Display), or a projector. In addition to the display device 111, other output peripheral devices can comprise components such as speakers (not shown) and a printer (not shown) which can be connected to the computer 101 via Input/Output Interface 110.

The computer 101 can operate in a networked environment using logical connections to one or more remote computing devices 114a, b, and c. By way of example, a remote computing device can be a personal computer, portable computer, a server, a router, a network computer, a peer device or other common network node, and so on. Logical connections between the computer 101 and a remote computing device 114a, b, and c can be made via a local area network (LAN) and a general wide area network (WAN). Such network connections can be through a network adapter 108. A network adapter 108 can be implemented in both wired and wireless environments. Such networking environments are conventional and commonplace in offices, enterprise-wide computer networks, intranets, and the Internet 115.

For purposes of illustration, application programs and other executable program components such as the operating system 105 are illustrated herein as discrete blocks, although it is recognized that such programs and components reside at various times in different storage components of the computing device 101, and are executed by the data processor(s) of the computer. An implementation of software 106 can be stored on or transmitted across some form of computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any method or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems of embodiments described herein can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

### Apparatus

In one embodiment, an Arrington ViewPoint Eye Tracker (Arrington Research, Inc, Scottsdale, Ariz.), or similar, device executing applicable software can be used to record eye movements and pupil diameter. While the Arrington ViewPoint Eye Tracker was used in the study provided herein, the methods and systems of embodiments described herein can be implemented with any device capable of monitoring a plurality of oculomotor measurements including but not limited to PD, rate of eye blinks, and gaze direction position. Other oculomotor measurements can include a number of fixations, total reading time, first fixation duration, second pass duration, probability of regressing into a region of interest, probability of regressing out of a region of interest, mean saccade duration, mean saccade distance, mean eye-screen distance. In one embodiment, the eye tracker can be affixed to a pair of lens-less plastic glasses. Viewing can be binocular, or can be monocular recording either the right or left eye.

In one embodiment, software, such as Eyelab 3.0 (Webb, A. K., Honts, C. R., Kircher, J. C., Bernhardt, P., Cook, A. E. (2008). Effectiveness of pupil diameter in a probable-lie comparison question test for deception), incorporated herein by reference), can be executed on an electronic device (e.g., computer 101 of FIG. 1a) to present stimuli to the subject, and collect, edit, and analyze the oculomotor data. The stimuli can be displayed on a plurality of monitor screens such as CRT or LCD (e.g., display device 111 of FIG. 1a). In one embodiment, the Eyelab, or similar software, can be configured to communicate with the Arrington ViewPoint Eye Tracker software via functions in Arrington's software development kit (SDK). Both Eyelab and Viewpoint programs can then be concurrently operated on a computer or network of computers, such as computer 101 of FIG. 1a.

In another embodiment, the PD or other oculomotor data can be imported into a computer program for psychophysiological research such as CPSLAB 10 (Scientific Assessment Technologies, Inc, Salt Lake City, Utah). In this embodiment, artifacts in the PD recordings caused by eye blinks can be tallied and edited from the recordings while the data are imported into CPSLAB. The tallying and editing can be performed simultaneously as the data are being recorded or can be done after the data has been recorded.

### Test Items

In one embodiment, the test items are presented in written form. Written form as used herein is any medium presented to that requires the subject to read the test items. Written medium may be paper, electronic, or any combination thereof.

All subjects are told to answer all items as quickly and accurately as possible to avoid appearing deceptive. No other cues are giving regarding what is defined as quickly or accurately, but rather left to the subjective interpretation of the subjects. In this way, a deceptive subjects' interpretation of these terms and subsequent behavior during the test can be compared to truthful subjects' performance during the test that received similar instructions. The variance between deceptive and truthful subjects can be used to differentiate between truthful and deceptive subjects.

In one embodiment, multiple test items can be administered to the subject with an equal distribution of items pertaining to the issue in question (also referred to as a relevant issue or issue of relevance), items pertaining to neutral issues, and items pertaining to a comparison issue (a topic that is comparable or equal to the relevant issue in perceived importance to a truthful test subject). Focused items for these three categories can be evenly distributed or staggered such that no two items from the same category appear in succession and do not reappear until an item for each of the other two categories is presented. By staggering the items, data collected from a previously deceptive response by the test subject concerning an item of interest will be less likely to interfere with the next item of interest.

The valence of the item (worded positively: “I did take the $20,” or worded negatively: “I did not take the $20”) can be balanced. Additionally, the appearances of key words in the items for the behavior in question and for the neutral items can be controlled. Key words can be those that are related to the issue in question. For example, in questioning about a theft of $20 from a secretary's purse, key words can be twenty dollars, wallet, purse or secretary to name but a few. Another example of key words can be professor, disk, exam or computer when questioning the theft of an exam disk. Key words are terms that are strongly associated with a crime and that would quickly alert the subject as to the content of the test item. It is possible that oculomotor measures taken during just reading of the key word may be predictive of deception. There is only one key word or phrase per item, and each key word or phrase appears an equal number of times across items.

Examples of true and false test items used in one embodiment of the present invention are shown in Table 1, below, but it is to be appreciated that these test items may be altered to suit the particular use of the technology. The length of test items (number of characters) can be equated within and between neutral, relevant, and comparison topic areas, and raw oculomotor measures can be expressed as rates to control for differences in statement length (e.g., number of fixations per character). Discriminating measures may include the mean response to neutral statements, the difference between the mean response to neutral statements and the mean response to relevant and comparison statements, and the difference between the mean response to relevant and comparison statements.

### Dependent Measures

During the subject's response to test items responds, single or multiple dependent oculomotor responses can be measured. Oculomotor herein is defined as any measure of pupil size, horizontal gaze location, vertical gaze location, torsion, convergence, or eye closures (blinks), or any measure derived from said measures (e.g., saccade velocity, number of eye fixations, first pass reading time). Oculomotor measures include but are not limited to any measure of pupil size, horizontal gaze location, vertical gaze location, torsion, convergence, blink rate, or blink magnitude, or any measure derived from said measures (e.g., saccade velocity, number of eye fixations, first pass reading time). Each channel of oculomotor data can be measured and stored in computer memory by the eye tracking device from 30 to 240 times per second (Hz).

In one embodiment, pupil diameter (PD) can be one of a plurality of oculomotor and behavioral dependent variables of interest in determining deception. PD response curves can be computed for each test item. In one embodiment, the response curve can begin when the item is presented and ends, for example, four seconds later. The original 30 Hz sampling rate can, for example, be reduced to 10 Hz by calculating a mean for each successive set of three samples. This procedure yields 40 data points for each item (4 s at 10 Hz). The first data point can be subtracted from every subsequent data point in the response curve to calculate deviations from initial level.

In one embodiment, two features can be extracted from the PD response curve—peak amplitude and area under the evoked PD response curve. Peak amplitude can be computed by identifying high and low points in the response curve and computing the difference between each low point and every succeeding high point. Peak amplitude is the greatest difference. Response onset is defined as the low point from which peak amplitude is computed. Area under the curve is the area under the response curve from response onset to the point at which the response returns to the initial level or to the end of the 4-s sampling interval, whichever occurs first.

In another embodiment, item blink rate and next item blink rate can be one of a plurality of dependent variables of interest in determining deception. Results from the study provided herein indicate that subjects inhibit their reading behavior when responding deceptively. Generally, subjects blink less often when they respond deceptively. In addition, there is an increase in the number of blinks on the item that follows an item answered deceptively as the subject attempts to recover from the threat posed by the prior item. As used herein, blink rate is the number of blinks per second. Blink rate is computed for each item (item blink rate) and for the item that followed (next item blink rate).

In another embodiment, fixations can be one of a plurality of dependent variables of interest in determining deception. Fixations can be determined from the data files produced by the eye tracking device (e.g., Arrington device discussed above) by identifying a sequence of samples in which the eye showed little movement for a minimum duration. In the study provided herein, a minimum duration of 100 ms was used. However, this duration is not required by the methods and systems of embodiments described herein and the duration may vary based on further calibration. The start of a fixation can be determined if the samples within the minimum duration window are within a standard deviation or predefined distance of each other. In the exemplary study described herein, a standard deviation of 0.5 was used, which means that for this example, the standard deviation of samples within the minimum duration window had to be less than 0.5 degrees of visual angle from the mean eye position. However, this standard deviation is not required by the methods and systems of embodiments described herein and may vary based on methods known to persons of ordinary skill in the art of statistical analysis such as, for example, range, interquartile range, variance, etc. In this exemplary embodiment, three sequential samples greater than one standard deviation from the running average fixation position can indicate the end of the fixation. The mean vertical position, mean horizontal position, and the duration of each fixation can be calculated once the points to be included in the fixation are identified. Once the series of samples that make up a fixation (e.g., fall within 0.5 degrees of visual angle of each other for at least 100 ms) are known, the mean horizontal position, the mean vertical position, the mean pupil diameter, the mean eye-screen distance, etc of the samples that are included in that fixation can be calculated. For the focused items, RT can be the time in seconds from the appearance of the item on the screen to a button press response by the subject.

In yet another embodiment, proportion wrong for a particular item type (neutral, comparison, relevant) can be computed by dividing the number of incorrect responses by the number of items. The number of fixations can be the number of fixations in an area of interest. First pass duration can be the sum of all fixation durations in an area of interest before the eye fixated outside the area of interest. Additionally, second pass duration can be the sum of all fixation durations in an area of interest after the first time the eye fixated outside the area of interest. Furthermore, reread duration can be the sum of all leftward eye movement fixation durations in an area of interest. This measure can be computed to assess rereading done by the subject whether or not the eye fixated outside the area of interest.

In another embodiment, response time can be one of a plurality of dependent variables of interest in determining deception. For the T/F items, RT is the time in ms from the appearance of the item on the screen to a button press response from the subject.

In another embodiment, reading duration can be one of a plurality of dependent variables of interest in determining deception. Multiple measures of reading duration can be calculated. For example, a first pass and second pass duration can be calculated. A first pass duration may be calculated as the sum of all fixation durations in an area of interest before the eye fixated outside the area of interest. A second pass duration may be calculated as the sum of all fixation durations in an area of interest after the first time the eye fixated outside the area of interest. In one embodiment, the area of interest may be defined as a rectangular area on the computer screen that encompasses the test item; it begins at the first character of the test item, ends at the last character of the test item, and has a height of about 2 degrees of visual angle.

In another embodiment, reread duration can be one of a plurality of dependent variables of interest in determining deception. In one example, reread duration may be calculated as the sum of all leftward eye movement fixation durations in an area of interest. While other methods are contemplated by one skilled it the art, reread measure may be computed to assess rereading done by the subject whether or not the eye fixated outside the area of interest.

While the present invention used the dependent measures described, one skilled in the art will appreciate that other dependent measures could be used. For example, it is contemplated that the dependent measures of the present invention can be combined with well-known dependent measures, whether or not currently used for credibility assessment, including blood pressure, pulse rate, respiration, breathing rhythms/ratios, skin conductivity or other responses produced during the presentation of test items, and these additional measures might be taken from sensors attached to the subject or remotely Moreover, the results of other credibility assessment tools such as a polygraph collected before or after the oculomotor test may be combined the results of the oculomotor test for an overall assessment of the examinee's veracity.

### Independent Variables

Single or multiple independent variables can also be included as part of the credibility assessment and ultimate analysis of the data. The independent variables can vary widely. In the present study multiple independent measures were studied, including item difficulty and test subject motivation, sex, age and ethnicity.

In another embodiment, motivation can be one of a plurality of independent variables of interest that can affect the accuracy of the deception test. Motivation can be a factor that can affect how people respond when they answer items in a screening situation or criminal investigation. Motivation was manipulated in the present study by offering subjects a monetary bonus to convince the examiner of their innocence. One skilled in the art will appreciate that that motivation may be manipulated using other means (e.g., a promise for a lighter sentence in a law enforcement setting, threat of punishment or imprisonment).

In another embodiment, item difficulty can be one of a plurality of independent variables. Hiding guilt can be difficult, and can require cognitive effort and self-control to suppress the truth, create the lie and make the correct response. In the present study, half of the subjects answered both difficult and easy items, and the remaining subjects answered only easy items. Difficult items included a relative clause (e.g., I am innocent of taking the item that was in the purse.) and easy items did not contain a relative clause. Research has demonstrated that sentences with relative causes are syntactically complex, and it can be more difficult to integrate information in a sentence as the number of phrases and clauses in the sentence increases. One skilled in the art will appreciate that that item difficulty may be manipulated using other forms of syntactic complexity, at the phrase, clause, or sentence level.

One skilled in the art will appreciate that independent variables may be used alone or in combination, whether or not they are disclosed herein or have previously been using in credibility assessment. Moreover, it may be desirable to manipulate independent variables and establish the particular set of conditions that maximize overall accuracy of classification. Other independent variables include but are not limited to reading ability, nationality, culture, and ethnicity.

### Analyzing Data

According to embodiments described herein, once the oculomotor data are collected dependent and independent measures are used to calculate the probability that the subject is providing a deceptive response. It is appreciated that one skilled in the art could use any number or combination of dependent and independent variables can be used in a linear or nonlinear discriminant or other statistical function for credibility assessment purposes. Any well known method for developing discriminant functions, including linear or quadratic discriminant analysis logistic regression analysis or predictive data mining techniques (e.g., bagging, boosting, stacking, meta-learning), could be used that makes optimal use of multiple oculomotor and behavioral measures for accurate discrimination of truthful and deceptive subjects.

In particular, for example, an increase in PD while responding to an item can indicate an increased likelihood of deceptive behavior. Furthermore, a decrease in response time while responding to an item can indicate an increased likelihood of deceptive behavior. Additionally, a decrease in the eye blink rate while responding to an item can be an indication of deceptive behavior. In one embodiment of the methods and system, one or more of the dependent and independent measures mentioned herein can be used to generate a discriminant function that is diagnostic of guilt or innocence.

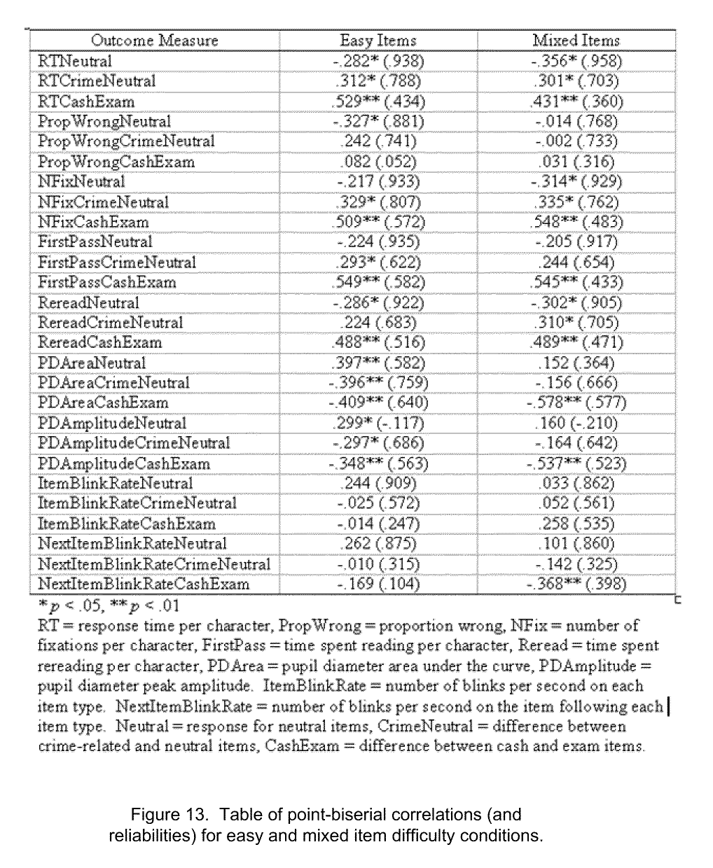

The dependent measures in FIG. 13 tagged with * or ** are statistically significant. That is, these dependent measures reliably discriminate between truthful and deceptive subjects. It is appreciated by one skilled in the art that it may be desirable to include even measures that do not show statistical significance in a discriminant function because the measures may work well in combination with other measures in the discriminant function. Measures that are not significantly correlated with guilt might, for example, add to the accuracy of a discriminant function by filtering noise from one or more other variables in the discriminant function. Such measures are sometimes called ‘suppressor’ variables because they suppress noise in other variables and make those variables better predictors of, for example, guilt.

It is to be appreciated that each discriminant function can and should be calibrated in a population of subjects similar to intended use population. That is, the discriminant function should be derived from a population that matches the intended population in all respects, including age, sex, nationality, language, reading ability and other independent variables. Likewise, the magnitude or diagnostic value of one or more dependent measures may be altered or moderated by independent variables. In other words, it is possible, even likely, that the discriminant function intended for the same purpose will differ depending on the characteristics of subjects or settings. The classifiers for different populations or settings may contain different subsets of dependent measures, or the classifiers may use the same measures but may weigh them differently to optimize the discrimination between truthful and deceptive subjects for a particular application (e.g., visa screening versus pre-employment screening versus periodic testing of current employees with access to the nation's secrets). It is understood by one skilled in the art that the techniques used herein can be adopted to these and other situations to optimize the diagnostic value of the credibility assessment for various purposes.

FIG. 1b provides a block diagram of some of the components of a system that can be used to determine the probability of a deceptive response, according to one embodiment. As shown, according to one embodiment, one or more sets of test items 120, 121, 122, 123, 124 can be ordered for display by an organizing device 130. In one aspect, the organizing device can be a computer such as, for example, the first computer 140 as shown in FIG. 1b. In other aspects, the organizing device 130 can be a separate computer or processor. The ordered test items 120, 121, 122, 123, 124 can then be displayed by a first computer 140 (e.g., computer 101 of FIG. 1a executing software, such as the Eyelab, or similar, software discussed above) on an operably connected monitor 141. A test subject 150 can then view the ordered test items on the monitor 141 and respond with a focused answer. An eye tracking device 142 (e.g., the Arrington ViewPoint Eye Tracker, discussed above; computer 101 of FIG. 1a executing eye tracking software; etc.), operably connected to the first computer, can monitor a plurality of eye movements as the test subject 150 views a test item from the plurality of ordered test items. A second computer 143 (e.g., computer 101 of FIG. 1a), operably connected to the eye tracking device 142 can receive data from the eye tracking device 142 and analyze the data using statistical analysis tools to determine the probability that the test subject 150 is being deceptive in responding to the plurality of test items. While shown as separate entities, as one of ordinary skill in the art will recognize in light of this disclosure, the functionality of one or more of the organizing device 130, first computer 140, eye tracking device 142, and/or second computer 143 can be combined into a single computer system, or similar electronic device. For example, according to one embodiment, the functionality of the organizing device 130 can be incorporated into the first computer 140.

In one embodiment, data from the eye tracking device 142 can be recorded for later analysis. In another embodiment, analysis of the eye tracking device 142 data can be an on-going process as the test subject responds to the ordered test items. In another aspect, this approach can be used with other lie detection techniques known to one of ordinary skill in the art such as the probable-lie comparison technique, which uses unfocused (ambiguous) test questions as a basis of comparison to focused (crime relevant) questions.

### Diagnostic Classification

The diagnostic classifications and diagnostic cutoffs are, within the boundaries of the selected discriminant function, arbitrary. In embodiments of the present invention several common statistical measures can be used. Sensitivity and specificity are statistical measures of the performance of a binary classification test (i.e., guilty or not guilty) well known in the art. Sensitivity measures the proportion of actual positives that are correctly identified as such (e.g., the percentage of guilty subjects who are correctly identified as being guilty). Specificity measures the proportion of negatives that are correctly identified (e.g., the percentage of innocent subjects who are correctly identified as being innocent). These two measures are closely related to the statistical concepts of type I and type II errors. A theoretical, optimal prediction can achieve 100% sensitivity (i.e., identify all guilty subjects in a population of guilty and innocent subjects) and 100% specificity (i.e., identify all innocent subjects in a population of guilty and innocent subjects).

For any binary classification test, there is usually a trade-off between the measures. For example, in an airport security setting it may be desirable to set the scanners to trigger on low-risk items like belt buckles and keys (low specificity), in order to reduce the risk of missing objects that do pose a threat to the aircraft and those aboard (high sensitivity). This trade-off can be represented graphically using a receiver operator curve (ROC) curve. Dependent on the desired risk level the sensitivity and specificity may be set at point along the ROC curve. The accuracy (ACC) used to describe the performance of the present invention is the mean proportion correct (i.e., (sensitivity+specificity)/2).

Other closely related measures are positive and negative predictive value. The positive predictive value, or precision rate, or post-test probability of disease, is the percentage of subjects with positive classifications (e.g., guilty) who are correctly classified. Conversely, the negative predictive value is the percentage of subjects with negative classifications (e.g., innocent) who are correctly classified.

### EXAMPLES

The following examples are put forth so as to provide those of ordinary skill in the art with a complete disclosure and description of how the compounds, compositions, articles, devices and/or methods of embodiments described herein are made and evaluated, and are intended to be purely exemplary and are not intended to limit the scope of the methods and systems. Efforts have been made to ensure accuracy with respect to numbers (e.g., amounts, temperature, etc.), but some errors and deviations should be accounted for. The examples described herein describe the usage of specific devices such as the Eyelab 3.0 and CPSLAB 10. It is to be appreciated that the devices described herein are not the only devices contemplated for providing the desired functions. Additionally, the examples described herein describe items given to test subjects. It is to be appreciated that the items herein are not the only items contemplated and that the items will vary depending on the subject matter(s) of interest. In the following example, guilt, motivation, and item difficulty were manipulated in the present study to determine their effects on oculomotor and behavioral measures of deception.

While the methods and systems have been described in connection with embodiments and specific examples, it is not intended that the scope be limited to the particular embodiments set forth, as the embodiments herein are intended in all respects to be illustrative rather than restrictive.

### Example 1

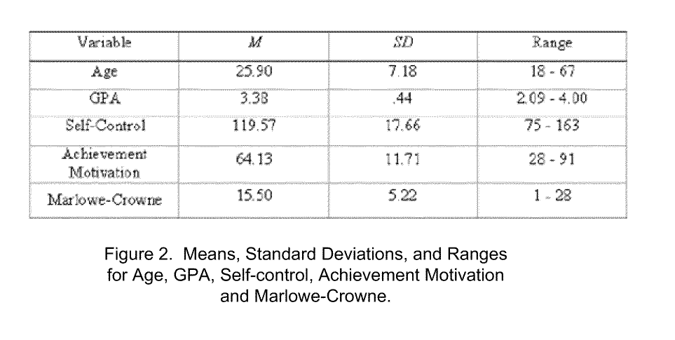

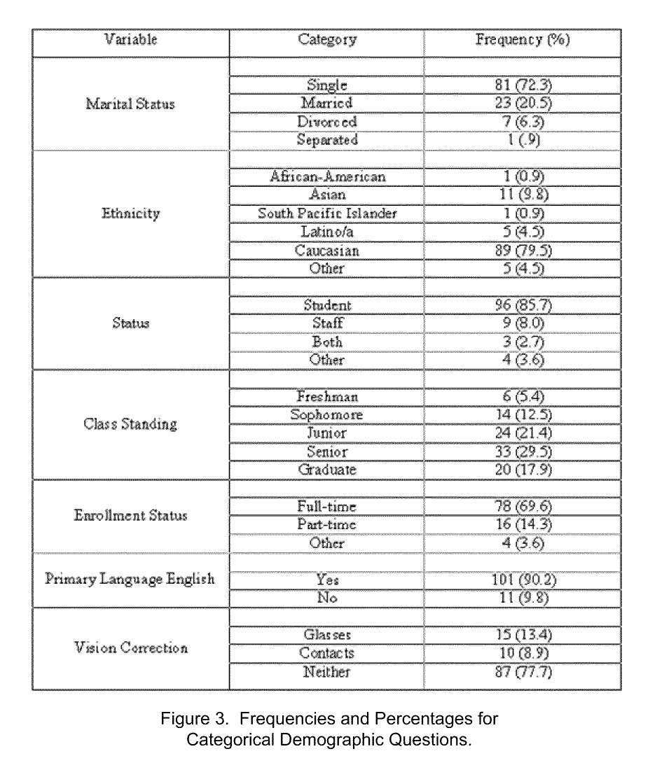

One hundred thirty-six subjects were recruited from the general University of Utah population. Recruitment flyers were posted on campus that advertised an opportunity to earn $30 and a possible bonus for participation in a psychological experiment. The flyer stated that potential participants must be student or staff and not need corrective lenses for reading. Of these 136 subjects, 8 chose not to participate after learning of their experimental condition, 5 did not follow instructions, 9 had poor or incomplete data, and 2 were lost due to experimenter error. This resulted in a sample size of 112 subjects. Demographic information obtained from subjects is illustrated in FIG. 2 and FIG. 3.

The design was a 2×2×2×2×(3×5) mixed design. The between-subjects variables were guilt (guilty and innocent), motivation ($30 and $1), item difficulty (mixed with both easy and difficult items and easy items only), and sex (male and female). The two within-subject factors were question type (neutral, cash, and exam) and repetition (5 repetitions of the T/F items). Time also was included as a within-subjects variable for the PD analyses. There were 40 levels for the time variable (10 Hz samples×4 seconds). There were 7 subjects in each of the 16 cells of the between-groups portion of the design for a total of 112 subjects.

Subjects answered 48 test items, and the 48 test items were repeated five times in different orders. All subjects received the same random order. Sixteen items pertained to the theft of the $20, 16 pertained to the theft of the exam, and 16 were neutral items. The items were arranged such that no two items from the same category appeared in succession. Half of the subjects received a mixed set of items that contained both easy and difficult items, and half received only easy items. The correct (nonincriminating) answer was true for 8 of the 16 items in each category and False for the remaining 8 items in each category. The valence of the item (worded positively: “I did take the $20,” or negatively: “I did not take the $20”) also was balanced for subjects in the mixed difficulty condition. Difficult items included a relative clause. Appearances of key words in the cash and exam items in both the easy and mixed conditions also were controlled. The key words in the cash items were twenty dollars, wallet, purse, and secretary. The key words in the exam items were professor, disk, exam, and computer. Only one key word appeared in each item, and each key word appeared twice within the set of 8 true and 8 false items in both the easy and mixed conditions. Additionally, each key word appeared only once in the True/Positive, True/Negative, False/Positive, and False/Negative conditions in the mixed set of items for both cash and exam items. The test items are presented in Table 1, below.

TABLE 1
T/F ITEMS

Mixed Items
Neutral Items
True/Positive
I was born prior to the year 1990.
The sky is blue on sunny days.
I attend a university that is in the United States.
The snow that accumulates in the winter melts in the spring.
True/Negative
I am reading this on a day other than Sunday.
Polar bears do not roam freely in Mexico.
I have eyes that are most definitely not the color orange.
The season that follows summer is not spring.
False/Positive
I am reading this sentence on Mar. 12, 2002.
San Francisco is in the state of Nevada.
I drive a car that runs on only air and water.
The Congo is a country that I visit several times a week.
False/Negative
I have never listened to radio or watched TV.
Whales do not live in any of the world's oceans.
I reside in a city that is not located in the state of Utah.
Trees that grow in the forest are never harvested for lumber.
Cash Items
True/Positive
I was uninvolved in the theft of the twenty dollars.
The wallet in the office was untouched by me.
I am innocent of taking the item that was in the purse.
The claim that I stole from a secretary is incorrect.
True/Negative
I did not take anything from the wallet in the office.
The theft involving the purse was not my fault.
I did not take anything that was in the secretary's office.
The twenty dollars that was in the office is not in my possession.
False/Positive
I am guilty of taking something from the secretary's office.
The reason the twenty dollars is gone is because I took it.
I admit to stealing the item that was in the purse.
The claim that I tampered with the wallet in the office is valid.
False/Negative
I am not innocent of stealing the twenty dollars.
The wallet was not recently tampered with by anyone but me.
I did not leave the office until I had taken the item that was in the
purse.
The claim that I did not take anything from the secretary is incorrect.
Exam Items
True/Positive
I took nothing from the professor's office.
The disk was untouched by me.
I left alone the information that was on the computer.
The exam that was in the office was left alone by me.
True/Negative
I did not take the exam from the office.
The information was not copied to a disk by me.
I never copied the information that was on the computer.
The claim that I copied the professor's information is not correct.
False/Positive
I made a copy of the professor's information.
The disk in my possession contains the copied information.
I have a copy of the information that was on the computer.
The exam that was in the office is in my possession.
False/Negative
I am not innocent of copying the information to a disk.
The loss of the exam is no one's fault but mine.
I did not pass up the chance to copy the information that was on the
computer.
The claim that I made a copy of the professor's information is not wrong.
Easy Items
Neutral Items
True
I was born prior to the year 1990.
The sky is blue on sunny days.
Cats and dogs are often kept as pets.
Dinosaurs used to roam the earth.
I am reading this on a day other than Sunday.
Polar bears do not roam freely in Mexico.
Global warming is a concern for many people.
Large SUVs often get lower gas mileage than newer compact cars.
False
I am reading this sentence on Mar. 12, 2002.
San Francisco is in the state of Nevada.
There are only 35 states in the United States.
Road construction is fast and convenient for motorists.
I have never listened to radio or watched TV.
Whales do not live in any of the world's oceans.
Trees are never harvested for lumber.
Morbid obesity is not a health concern in the United States.
Cash Items
True
I was uninvolved in the theft of the twenty dollars.
The wallet in the office was untouched by me.
I did not take anything from the wallet in the office.
The theft involving the purse was not my fault.
The secretary's property was not stolen by me.
I am innocent of taking the secretary's property from the office.
I had nothing to do with the theft of the twenty dollars.
The article from the purse was not stolen by me.
False
I am guilty of taking something from the secretary's office.
The reason the twenty dollars is gone is because I took it.
I am not innocent of stealing the twenty dollars.
The wallet was not recently tampered with by anyone but me.
The item from the purse is hidden on my person.
I removed something from the purse in the office.
The secretary's property was stolen by me.
I know what happened to the item missing from the wallet.
Exam Items
True
I took nothing from the professor's office.
The disk was untouched by me.
I did not take the exam from the office.
The information was not copied to a disk by me.
The loss of the professor's information is not my fault.
The information from the computer is not in my possession.
I did not take anything from the computer in the office.
I am not guilty of taking the exam from the office.
False
I made a copy of the professor's information.
The disk in my possession contains the copied information.
I am not innocent of copying the information to a disk.
The loss of the exam is no one's fault but mine.
I took the information from the computer in the office.
The professor's information is missing because of me.
The missing exam is in my possession.
I copied the information from the computer.

### Questionnaires

Subjects completed several questionnaires, one of which was a demographic questionnaire (Table 2), below.

TABLE 2
Demographic Questionnaire

Participant ID #

| | |
|---|---|
| 1. | Age: |
| 2. | Sex: (circle one) Male Female |
| 3. | Marital status: (circle one) |
| | Single |
| | Married |
| | Divorced |
| | Widowed |
| | Separated |
| 4. | Racial/Ethnic Origin: (circle one) |
| | African American |
| | Asian |
| | South Pacific Islander |
| | Latino/a |
| | American Indian |
| | Middle Eastern |
| | Caucasian |
| | Other (please explain): |
| 5. | What is your status? (circle one) |
| | Student |
| | Staff |
| | Other |
| 6. | If you are a student, what is your college |
| | major? |
| 7. | If you are a student, what is your class standing? (circle one) |
| | Freshman |
| | Sophomore |
| | Junior |
| | Senior |
| | Graduate |
| 8. | If you are a student, what is your enrollment status? (circle one) |
| | Full-time |
| | Part-time |
| | Other (please explain): |
| 9. | If you are a student, what is your current GPA? |
| 10. | If you are not a student, what is the highest level of school or |
| | degree you have completed? (circle one) |
| | High school |
| | Trade school |
| | Associate's degree |
| | Bachelor's degree |
| | Master's degree |
| | Professional degree |
| | Doctorate degree |
| 11. | Is English your primary language? (circle one) Yes No |
| | If you circled No, what is your primary |
| | language? |
| 12. | Do you wear any of the following for vision correction for reading? |
| | (circle one) |
| | Glasses |
| | Contacts |
| | Neither |

Subjects also completed the Self-Control Scale. The Self-Control Scale consists of 36 items and is designed to assess individual differences in self-control. Because motivation was a key part of this experiment, subjects completed a Cassidy-Lynn Achievement Motivation Questionnaire. The questionnaire was used to assess their motivations to determine if subjects are motivated by money, or if they complete tasks for their intrinsic value. Some of the items on the achievement motivation questionnaire are items that some people may respond to in a socially desirable manner. Prior studies found moderate correlations between the Self-Control Scale and social desirability. To assess a subject's tendency to respond in a socially desirable manner, the Marlowe-Crowne Social Desirability Questionnaire also was administered.

Procedure

Interested subjects called a secretary to set up an appointment. The secretary ensured subjects were 18 years of age or older, university students or staff, and proficient at speaking and reading English. Subjects were emailed preliminary instructions and a map of campus two or three days prior to their scheduled appointment. Subjects were called the day before their appointment to remind them of their appointment and to ask them to get adequate sleep the night before and to refrain from caffeine for a few hours prior to their appointment. Prior experience suggested caffeine makes it more difficult to calibrate the subject because the pupils are more constricted.

Subjects arrived alone at their appointment. Instructions in an envelope taped to the door instructed them to enter the room, read and sign the consent form, fill out the questionnaires in order, and take the consent form and questionnaires with them when they left, and to give the materials to the experimenter. The instructions also stated they would receive further instructions after completing the questionnaires. After reading and signing the consent form, subjects completed the demographic questionnaire, the Cassidy-Lynn Achievement Motivation Questionnaire, the Self-Control Scale, and the Marlowe-Crowne Social Desirability Scale. Another envelope attached to the back of the questionnaire packet instructed them to locate a cassette tape, listen to the cassette, and rewind and return the cassette to the location in which they found it. A printed copy of the cassette instructions was included in the envelope. A phone number was provided for subjects to call if they did not wish to participate.

Half of the subjects were in the guilty condition. Guilty subjects were instructed to go to a secretary's office on another floor of the building and ask the secretary where Dr. Laird's office was located. The secretary told the subject that there was no Dr. Laird in the building, and the subject thanked the secretary and left. The subject waited for the secretary to leave her office, then entered her office, found her purse, and removed $20 from a wallet in the purse and concealed the money. Subjects were told to prepare an alibi in case they were caught and not leave fingerprints. Subjects were told that they had no more than 20 min to commit the crime and report to the experimenter.

Half of the subjects were in the innocent condition and did not steal anything. They were told that some subjects had to steal something from an office, but that they were innocent subjects and did not have to steal anything. Innocent subjects were told to wait approximately 20 minutes before reporting to the experimenter. All subjects were told that there was another crime in which some subjects had to download an exam from a professor's computer onto a disk, but in actuality, no one committed that crime.

Half of the subjects were told that they would receive an additional $30 bonus (high motivation condition) in addition to the possible $30 advertised on the flyer if they were able to convince the examiner that they were innocent of both crimes. The remaining subjects were told that they would receive an additional $1 bonus (low motivation condition) if they were able to convince the examiner of their innocence.

Subjects reported to the experimenter after committing their crime or after an appropriate waiting period. The experimenter placed the eye tracker on the subject and then calibrated the equipment. Instructions and practice items then were presented to the subject in a black font on a grey background. Subjects began answering test items after they had answered 15 practice items. Subjects received practice items only on the first repetition. Items were presented on the screen one at a time. A T/F appeared on the right side of the screen to remind subjects of their response options. Subjects responded to the test items using buttons on the computer keyboard. After responding, a TRUE or FALSE (depending on the subject's response) appeared on the right side of the screen for 500 ms to indicate the response to the subject. The next item appeared automatically. Subjects answered 48 items in this manner.

The subject then completed an intervening task. The intervening task consisted of 24 T/F general world knowledge questions. The purpose of the intervening task was to minimize retention of the test items and answers. Subjects completed 5 repetitions of the test items and 4 repetitions of the intervening task items. Intervening task items were not repeated across repetitions and were not used to make decisions about the subject's veracity. Subjects took between 3 and 6 minutes to complete the test items and between 2 and 4 minutes to complete the intervening task. Subjects were told to answer all items as quickly and accurately as possible to avoid appearing deceptive.

Dependent measures for the test items were response time (RT), proportion wrong, number of fixations, first pass duration, reread duration, and area under the evoked PD response curve. After the fifth repetition of the 48 T/F items, subjects completed a final task that required about 5 minutes. Following that task, subjects were paid and debriefed. Subjects were told that their payment was based on their experimental condition. Guilty and innocent subjects in the low motivation condition were paid $31 ($30 base pay plus $1 bonus). Guilty and innocent subjects in the high motivation condition were paid $60 ($30 base pay plus $30 bonus). Subjects then were interviewed to assess any strategies they may have used and what they felt and thought while completing the tasks. The interview consisted of both multiple-choice and open-ended questions. The interview questions are presented in Table 3, below. Subjects were paid and debriefed prior to the interview in an attempt to ensure more honesty from the subject than might have been obtained if the subject had not been paid and still was trying to convince the experimenter of their innocence. After the interview, subjects were asked not to discuss details of the study with others and released.

TABLE 3
INTERVIEW QUESTIONS

Participant ID#
1. On a scale of 1 to 5 with 1 being not at all and 5 being extremely,
how anxious were you feeling at the beginning of the experiment?

| | | | | |
|---|---|---|---|---|
| 1 | 2 | 3 | 4 | 5 |
| Not at all | | | | Extremely |

2. On a scale of 1 to 5 with 1 being not at all and 5 being extremely,
how anxious are you feeling right now?

| | | | | |
|---|---|---|---|---|
| 1 | 2 | 3 | 4 | 5 |
| Not at all | | | | Extremely |

3. Your base pay was $30, and you were promised a $ bonus if
you could convince the examiner of your innocence. How important
was the $ monetary bonus to you?

| | | | |
|---|---|---|---|
| 1 | 2 | 3 | 4 |
| Not at all | Somewhat | Very | Extremely |

4. If you had been promised a $ bonus, would you have acted
differently? If so, how?
5. As you were answering the true and false items, were you more
concerned about how quickly you answered, if you answered
correctly, or equally concerned about both?

a. how quickly you answered
b. if you answered correctly
c. equally concerned about both

6. Which true and false items were you most concerned about?

a. items about the theft of the exam information
b. items about the theft of the $20
c. neutral items
d. all of the items
e. some combination

7. Did you develop any strategies to convince the experimenter
of your innocence as you were answering the true and false
items? Yes No
8. How would you teach someone else to beat the test? Is there
anything specific you would tell them?

Areas of Interest

An area of interest was defined for each T/F test item prior to the calculation of the dependent measures. The area of interest began with the first character of the item and ended after the period at the end of the item. First pass duration, second pass duration, number of fixations, and reread duration were computed for the fixations in each area of interest. RT, number of fixations, first pass duration, second pass duration, and reread duration were divided by the number of characters in an item to control for differing item lengths. Number of characters did not differ as a function of item difficulty, p>0.05, but did differ as a function of question type, p<0.05. Cash items were the longest (M=54.875, SD=8.059), followed by the exam (M=50.938, SD=10.665) and neutral items (M=45.906, SD=9.410).

### Fixations

Fixations were determined from the data files produced by the Arrington by identifying a sequence of samples in which the eye showed little movement for 100 ms. The start of a fixation was determined if the samples within the 100 ms time window were within 0.5 standard deviations of each other when measured in degrees of visual angle. Three sequential samples greater than one standard deviation from the running average fixation position indicated the end of the fixation. The mean vertical position, mean horizontal position, and the duration of each fixation were calculated.

### Response Time

For the T/F items, RT was the time in s from the appearance of the item on the screen to a button press response from the subject.

### Proportion Wrong

Proportion wrong for a particular item type (neutral, cash, exam) was computed by dividing the number of incorrect responses by the number of items (16).

### Number of Fixations

Number of fixations was the number of fixations in an area of interest.

### First Pass Duration

First pass duration was the sum of all fixation durations in an area of interest before the eye fixated outside the area of interest.

### Second Pass Duration

Second pass duration was the sum of all fixation durations in an area of interest after the first time the eye fixated outside the area of interest.

### Reread Duration

Reread duration was the sum of all leftward eye movement fixation durations in an area of interest. This measure was computed to assess rereading done by the subject whether or not the eye fixated outside the area of interest.

### Pupil Diameter

PD response curves were computed for each item. The response curve began when the item was presented and ended 4 s later. The original 30 Hz sampling rate was reduced to 10 Hz by calculating a mean for each successive set of three samples. This procedure yielded 40 data points for each item (4 s at 10 Hz). The first data point was subtracted from every subsequent data point in the response curve to calculate deviations from initial level. Two features were extracted from the PD response curve and are defined as follows:

Peak amplitude was computed by identifying high and low points in the response curve and computing the difference between each low point and every succeeding high point. Peak amplitude was the greatest difference. Response onset was defined as the low point from which peak amplitude was computed.

Area to full recovery was the area under the response curve from response onset to the point at which the response returned to the initial level or to the end of the 4-s sampling interval, whichever occurred first.

### Item Blink Rate and Next Item Blink Rate

Blink rate was the number of blinks per second. Blink rate was computed for each item (item blink rate) and for the item that followed (next item blink rate). A decrease in item blink rate may be thought of as an indicator of cognitive load, whereas an increase in next item blink rate may be viewed as a measure of relief.

Results

Significance for tests involving a repeated factor (repetition, question type, and time) used Huynh-Feldt corrections to degrees of freedom. Effects were significant at p<0.05 unless otherwise noted. Analyses were conducted on both second pass and reread duration. Results were similar for the two and because second pass is a special case of reread duration, only results for reread duration are reported.

Manipulation Check

Analysis of variance was performed on the interview question subjects answered at the end of their session regarding the importance of the monetary bonus. Guilt, motivation, item difficulty, and sex were included as factors. The monetary bonus was generally more important to subjects promised a $30 bonus for a truthful outcome (M=2.866, SE=0.112) than to subjects promised only $1 for a truthful outcome (M=1.750, SE=0.112), F(1, 96)=49.61, partial η²=0.341. The bonus was more important to males (M=2.473, SE=0.112) than to females (M=2.143, SE=0.112), F(1,96)=4.35, partial η²=0.043. There also was a guilt by item difficulty interaction for importance of the monetary bonus, F(1,96)=11.05, partial η²=0.103. The monetary bonus was generally most important to guilty subjects who received mixed items and least important to innocent subjects who received mixed items. Taken together, these results suggest the motivation manipulation affected perceptions of the importance of the monetary bonus.

The relationship between self-reports of the importance of the monetary bonus and scores on the acquisitiveness for money and material wealth subscale of the achievement motivation subscale also was examined. The correlation was not significant, r=0.161, p>0.05.

### Example 2

### Effects of Guilt, Motivation, and Item Difficulty on T/F Items

Repeated measures analyses of variances (RMANOVAs) were conducted on each dependent variable. For RT, proportion wrong, number of fixations, first pass duration, reread duration, and blink rates, the between-subjects factors were guilt, motivation, item difficulty, and sex, and the within-subjects factors were question type and repetition. For PD, the between subjects factors were guilt, motivation, item difficulty, and sex, and the within-subjects factors were question type, repetition, and time. The RMANOVA analyses contained more than 60 sources of variance. To simplify presentation of the results and because guilt was the manipulation of greatest interest, only main effects of guilt and guilt interactions are presented and discussed in the text. A tables that includes effect sizes for all statistically significant main effects and interactions for each dependent variable is presented in Table 4, below. Four-way and higher order interactions are reported but not discussed.

TABLE 4

| | EFFECT SIZE | | | | | | | |
|---|---|---|---|---|---|---|---|---|
| | | | | | | | | Next |
| | | | | First | | | Item | Item |
| | Response | Proportion | No. of | Pass | Reread | Pupil | Blink | Blink |
| Source | Time | Wrong | Fixations | Duration | Duration | Diameter | Rate | Rate |
| Guilt | .052 | .055 | | | .047 | | | |
| Motiv | | | | | | | | |
| Item | | | .047 | | | | | |
| Sex | | | | .038 | | | | |
| Rep | .567 | .124 | .540 | .282 | .505 | .042 | | |
| QT | .277 | .146 | .185 | .273 | .325 | .535 | | |
| Time | | | | | | .056 | | |
| Guilt × Motiv | | | | | | | | |
| Guilt × Item | | .046 | | | | | | |
| Guilt × Sex | | | | | | | | |
| Motiv × Item | .041 | | | | | | | |
| Motiv × Sex | | | | | | | | |
| Item × Sex | | | | | | | | |
| Rep × Guilt | | | | | | .034 | | |
| Rep × Motiv | | | | | | | | |
| Rep × Item | | | | | | | | |
| Rep × Sex | | | | | .026 | | | |
| Rep × QT | .068 | .044 | .069 | .086 | .040 | .071 | | |
| Rep × Time | | | | | | .017 | | |
| QT × Guilt | .142 | | .173 | .163 | .136 | .157 | | .044 |
| QT × Motiv | | | | | | .044 | | |
| QT × Item | .067 | .077 | .086 | .096 | .086 | | | |
| QT × Sex | | .047 | | | | | | |
| QT × Time | | | | | | .467 | | |
| Time × Guilt | | | | | | | | |
| Time × Motiv | | | | | | | | |
| Time × Item | | | | | | | | |
| Time × Sex | | | | | | | | |
| Guilt × Motiv × | | | | | | | | |
| Item | | | | | | | | |
| Guilt × Motiv × Sex | | | | | | | | |
| Guilt × Item × Sex | | | | | | | | |
| Motiv × Item × Sex | | | | | .040 | | | |
| Rep × Guilt × | | | | | | | .027 | |
| Motiv | | | | | | | | |
| Rep × Guilt × Item | | | | | | | | |
| Rep × Motiv × Item | | .026 | | | | | | |
| Rep × Guilt × Sex | | | | | | | | |
| Rep × Motiv × Sex | | | | | | | | |
| Rep × Guilt × | | | | | | | | .028 |
| Motiv | | | | | | | | |
| Rep × Guilt × Item | | | | | | | | |
| Rep × Motiv × Item | | | | | | | | |
| Rep × Guilt × Sex | | | | | | | | |
| Rep × Motiv × Sex | | | | | | | | |
| Rep × Item × Sex | | | | | | | | |
| Rep × QT × Guilt | | | | | | | | |
| Rep × QT × Motiv | | | | | | | | .021 |
| Rep × QT × Item | .024 | | .028 | | | | | |
| Rep × QT × Sex | | | | | | | | |
| Rep × QT × Time | | | | | | .043 | | |
| Rep × Time × Guilt | | | | | | | | |
| Rep × Time × | | | | | | | | |
| Motiv | | | | | | | | |
| Rep × Time × Item | | | | | | | | |
| Rep × Time × Sex | | | | | | .021 | | |
| QT × Guilt × Motiv | | | .034 | | | | | |
| QT × Guilt × Item | | | | | | | | |
| QT × Motiv × Item | | | | | | | | |
| QT × Guilt × Sex | | | | | | | .039 | |
| QT × Motiv × Sex | | | | | | | | |
| QT × Item × Sex | | | | | | | | |
| QT × Time × Guilt | | | | | | .104 | | |
| QT × Time × Motiv | | | | | | | | |
| QT × Time × Item | | | | | | | | |
| QT × Time × Sex | | | | | | | | |
| Time × Guilt × | | | | | | | | |
| Motiv | | | | | | | | |
| Time × Guilt × Item | | | | | | | | |
| Time × Motiv × | | | | | | | | |
| Item | | | | | | | | |
| Time × Guilt × Sex | | | | | | | | |
| Time × Motiv × | | | | | | | | |
| Sex | | | | | | | | |
| Time × Item × Sex | | | | | | | | |
| Guilt × Motiv × | | | | | | | | |
| Item × Sex | | | | | | | | |
| Rep × Guilt × | | | | | | | | |
| Motiv × Item | | | | | | | | |
| Rep × Guilt × | | | | | .028 | | | |
| Motiv × Sex | | | | | | | | |
| Rep × Guilt × Item × | | | | | | | | |
| Sex | | | | | | | | |
| Rep × Motiv × Item × | | | | | | | | |
| Sex | | | | | | | | |
| Rep × QT × Guilt × | | | | | | | | |
| Motiv | | | | | | | | |
| Rep × QT × Guilt × | | | | | | | | .020 |
| Item | | | | | | | | |
| Rep × QT × Motiv × | | | | | | | | |
| Item | | | | | | | | |
| Rep × QT × Guilt × | | | | | | | | |
| Sex | | | | | | | | |
| Rep × QT × Motiv × | | | | | | | | |
| Sex | | | | | | | | |
| Rep × QT × Item × | | | | | | | | |
| Sex | | | | | | | | |
| Rep × Time × Guilt × | | | | | | | | |
| Motiv | | | | | | | | |
| Rep × Time × Guilt × | | | | | | | | |
| Item | | | | | | | | |
| Rep × Time × | | | | | | | | |
| Motiv × Item | | | | | | | | |
| Rep × Time × Guilt × | | | | | | | | |
| Sex | | | | | | | | |
| Rep × Time × | | | | | | | | |
| Motiv × Sex | | | | | | | | |
| Rep × Time × Item × | | | | | | | | |
| Sex | | | | | | | | |
| Rep × QT × Time × | | | | | | .021 | | |
| Guilt | | | | | | | | |
| Rep × QT × Time × | | | | | | | | |
| Motiv | | | | | | | | |
| Rep × QT × Time × | | | | | | | | |
| Item | | | | | | | | |
| Rep × QT × Time × | | | | | | | | |
| Sex | | | | | | | | |
| QT × Guilt × Motiv × | | | | | | | | |
| Item | | | | | | | | |
| QT × Guilt × Motiv × | | | | .042 | | | | |
| Sex | | | | | | | | |
| QT × Guilt × Item × | | | | | | | | |
| Sex | | | | | | | | |
| QT × Motiv × Item × | | | | | | | | |
| Sex | | | | | | | | |
| QT × Time × Guilt × | | | | | | | | |
| Motiv | | | | | | | | |
| QT × Time × Guilt × | | | | | | | | |
| Item | | | | | | | | |
| QT × Time × Motiv × | | | | | | | | |
| Item | | | | | | | | |
| QT × Time × Guilt × | | | | | | | | |
| Sex | | | | | | | | |
| QT × Time × Motiv × | | | | | | | | |
| Sex | | | | | | | | |
| QT × Time × Item × | | | | | | | | |
| Sex | | | | | | | | |
| Time × Guilt × | | | | | | | | |
| Motiv × Item | | | | | | | | |
| Time × Guilt × | | | | | | .042 | | |
| Motiv × Sex | | | | | | | | |
| Time × Guilt × Item × | | | | | | | | |
| Sex | | | | | | | | |
| Time × Motiv × | | | | | | | | |
| Item × Sex | | | | | | | | |
| Rep × Guilt × | | | | | | | | |
| Motiv × Item × Sex | | | | | | | | |
| Rep × QT × Guilt × | | | | | | | | |
| Motiv × Item | | | | | | | | |
| Rep × QT × Guilt × | | .027 | | | | | | |
| Motiv × Sex | | | | | | | | |
| Rep × QT × Guilt × | | | | .027 | | | | |
| Item × Sex | | | | | | | | |
| Rep × QT × Motiv × | | | | | | | | |
| Item × Sex | | | | | | | | |
| Rep × Time Guilt × | | | | | | | | |
| Motiv × Item | | | | | | | | |
| Rep × Time × Guilt × | | | | | | | | |
| Motiv × Sex | | | | | | | | |
| Rep × Time × Guilt × | | | | | | | | |
| Item × Sex | | | | | | | | |
| Rep × Time × | | | | | | | | |
| Motiv × Item × Sex | | | | | | | | |
| Rep × QT × Time × | | | | | | | | |
| Guilt × Motiv | | | | | | | | |
| Rep × QT × Time × | | | | | | | | |
| Guilt × Item | | | | | | | | |
| Rep × QT × Time × | | | | | | | | |
| Motiv × Item | | | | | | | | |
| Rep × QT × Time × | | | | | | | | |
| Guilt × Sex | | | | | | | | |
| Rep × QT × Time × | | | | | | | | |
| Motiv × Sex | | | | | | | | |
| Rep × QT × Time × | | | | | | | | |
| Item × Sex | | | | | | | | |
| QT × Guilt × Motiv × | | | | | | | | |
| Item × Sex | | | | | | | | |
| QT × Time × Guilt × | | | | | | | | |
| Motiv × Item | | | | | | | | |
| QT × Time × Guilt × | | | | | | | | |
| Motiv × Sex | | | | | | | | |
| QT × Time × Guilt × | | | | | | | | |
| Item × Sex | | | | | | | | |
| QT × Time × Motiv × | | | | | | | | |
| Item × Sex | | | | | | | | |
| Time × Guilt × | | | | | | | | |
| Motiv × Item × Sex | | | | | | | | |
| Rep × QT × Guilt × | | | | | | | | |
| Motiv × Item × Sex | | | | | | | | |
| Rep × Time × Guilt × | | | | | | | | |
| Motiv × Item × | | | | | | | | |
| Sex | | | | | | | | |
| QT × Time × Guilt × | | | | | | | | |
| Motiv × Item × | | | | | | | | |
| Sex | | | | | | | | |
| Rep × QT × Time × | | | | | | | | |
| Guilt × Motiv × | | | | | | | | |
| Item | | | | | | | | |
| Rep × QT × Time × | | | | | | | | |
| Guilt × Motiv × Sex | | | | | | | | |
| Rep × QT × Time × | | | | | | | | |
| Guilt × Item × Sex | | | | | | | | |
| Rep × QT × Time × | | | | | | | | |
| Guilt × Motiv × | | | | | | | | |
| Item × Sex | | | | | | | | |
| Rep × QT × Time × | | | | | | | | |
| Guilt × Motiv × | | | | | | | | |
| Item × Sex | | | | | | | | |

Rep = repetition, Motiv = motivation, Item = item difficulty, QT = question type

Significant guilt by question type interactions were followed by contrasts to determine if there were differences between the crime and neutral items and between the cash and exam items within the guilty and innocent groups. Tests also were conducted to determine if the guilty and innocent groups differed on responses to neutral, cash, and exam items. A p-value of 0.01 was used for follow-up tests.

There were 11 subjects who reported that English was not their native language. Three of these subjects were in the guilty group and eight were in the innocent group. There was no significant difference in the proportion of non-English speakers in the guilty and innocent groups, p>0.05.

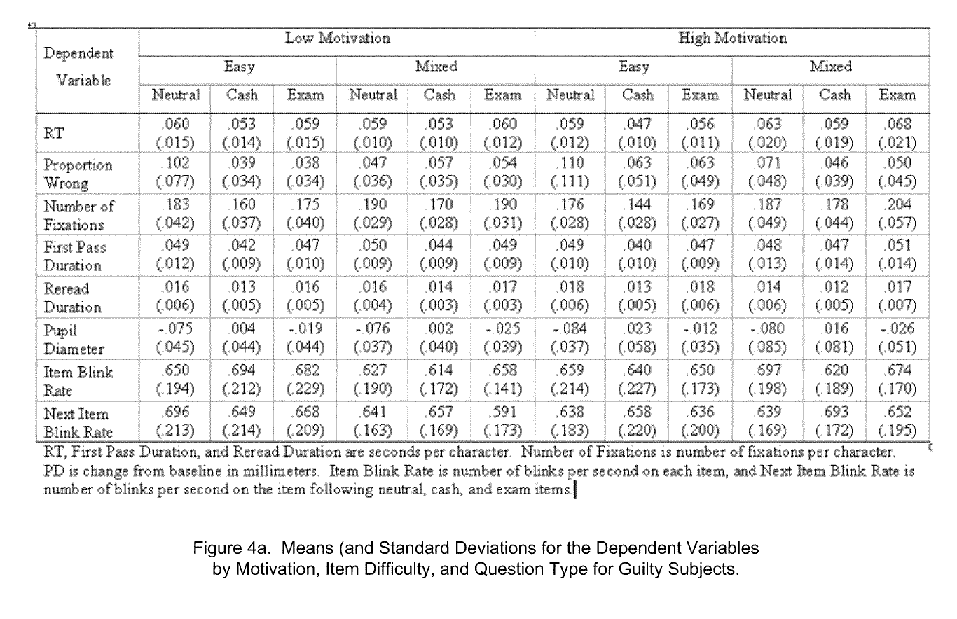

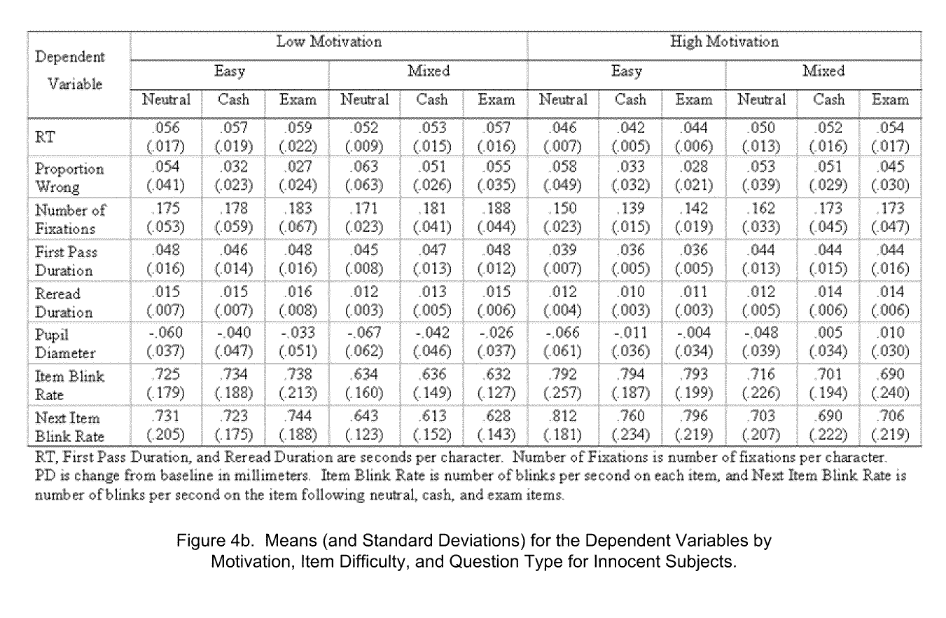

Means and standard deviations for the eight dependent variables are illustrated in FIG. 4a and FIG. 4b for guilty and innocent subjects, respectively. They are broken down by motivation, item difficulty, and question type. There were few interpretable effects for sex, so means and standard deviations were pooled over levels of sex.

### Response Time

The main effect of guilt was significant, F(1,96)=5.28. Guilty subjects generally took longer to respond (M=0.058, SE=0.002) than did innocent subjects (M=0.052, SE=0.002). The effect of guilt on RT was not moderated by motivation or item difficulty.

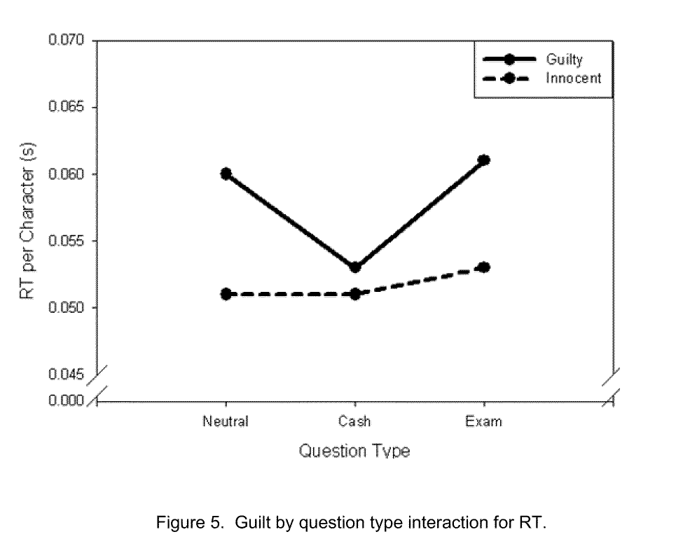

The guilt by question type interaction was significant, F(2,192)=15.89, and is illustrated in FIG. 5. For guilty subjects, RTs were generally longest for the exam items (M=0.061, SE=0.002), followed by the neutral items (M=0.060, SE=0.002), and the cash items (M=0.053, SE=0.002). For innocent subjects, RTs were nearly identical for the neutral and cash (Ms=0.051, SEs=0.002) items, and both were shorter than RTs to the exam items (M=0.053, SE=0.002). Follow-up tests indicated that guilty subjects generally responded more quickly to the crime-related items than to the neutral items, F(1,55)=17.28, partial η²=0.239, and responded more quickly to the cash items than to the exam items, F(1,55)=117.79, partial η²=0.682. Innocent subjects generally also responded more quickly to the cash items than to the exam items, F(1,55)=27.96, partial η²=0.337. Follow-up tests also indicated that guilty and innocent subjects differed in RT only on the neutral items, p<0.01. The guilt by question type interaction was not moderated by motivation or item difficulty.

### Proportion Wrong

The main effect of guilt was significant, F(1,96)=5.63. Guilty subjects tended to make more mistakes overall (M=0.062, SE=0.005) than did innocent subjects (M=0.046, SE=0.005).

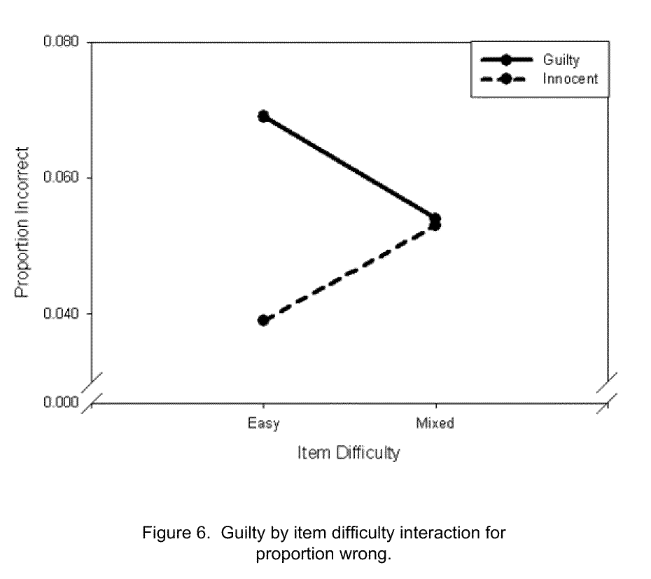

The guilt by item difficulty interaction was significant, F(1,96)=4.62, and is illustrated in FIG. 6. Guilty subjects in the easy item condition answered the most items incorrectly (M=0.069, SE=0.007), followed by guilty subjects in the mixed condition (M=0.054, SE=0.007), innocent subjects in the mixed condition (M=0.053, SE=0.007), and innocent subjects in the easy condition (M=0.039, SE=0.007).

The guilt by motivation by sex by question type by repetition interaction was significant, F(8,768)=2.64.

### Number of Fixations

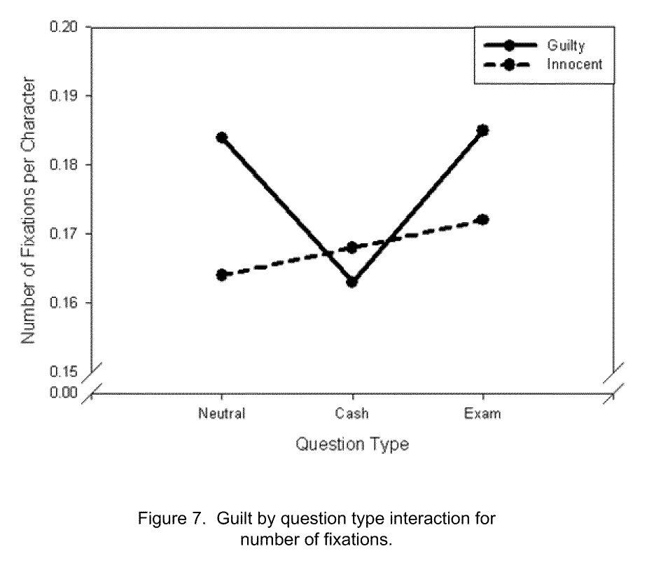

The guilt by question type interaction was significant, F(2,192)=20.03, and is illustrated in FIG. 7. Guilty subjects generally made similar numbers of fixations on the neutral and exam items and the fewest on the cash items. Follow-up tests indicated that guilty subjects generally made more fixations on neutral items than on crime-related items, F(1,55)=13.30, partial η²=0.195, and more fixations on exam items than on cash items, F(1,55)=99.59, partial η²=0.644. Follow-up tests also indicated that guilty and innocent subjects differed in number of fixations for the neutral items, p<0.01.

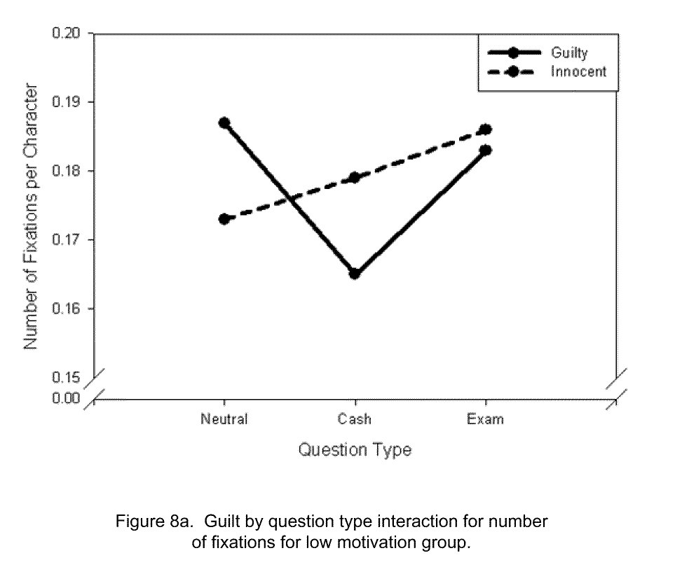

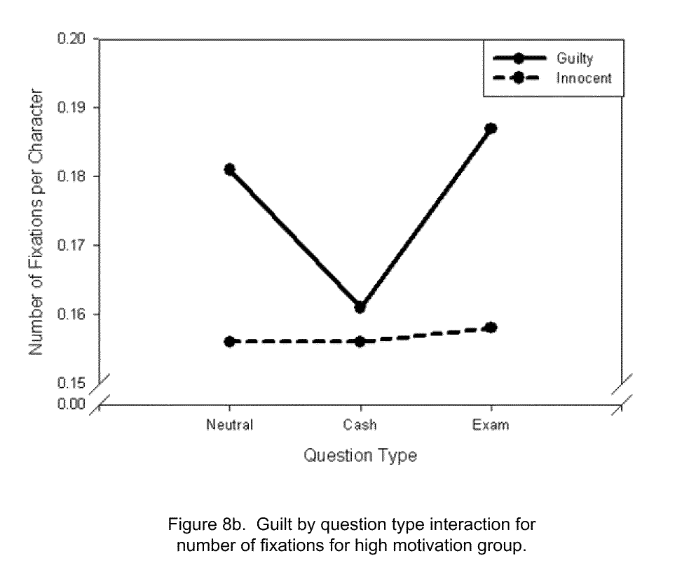

The guilt by motivation by question type interaction also was significant, F(2,192)=3.38. This effect is illustrated graphically in FIG. 8a and FIG. 8b. Guilty subjects generally made fewer fixations on the cash items than the neutral or exam items in both motivation conditions. Motivation had more effect on innocent subjects generally than guilty subjects. Innocent subjects in the low motivation condition made more fixations than did innocent subjects in the high motivation condition. Follow-up analyses indicated that the magnitude of the guilt by question type interaction was similar for both motivation groups (low: F(2,108)=11.21, partial η²=0.172; high: F(2,108)=11.11, partial η²=0.171).

### First Pass Duration

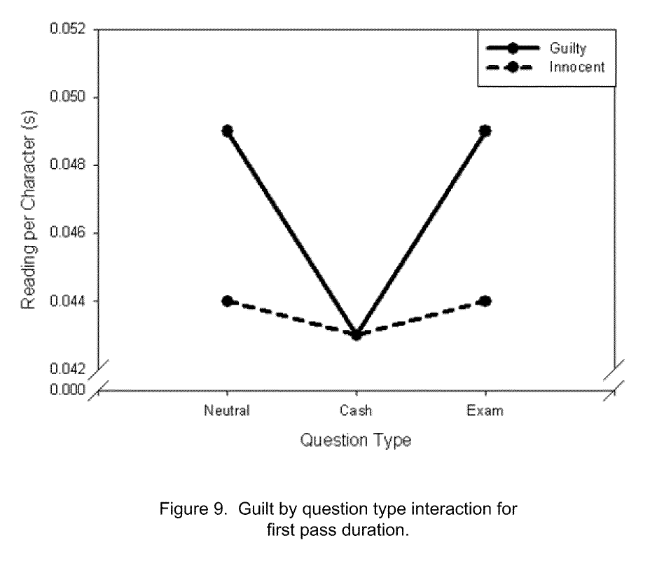

The guilt by question type interaction was significant, F(2,192)=18.69, and is illustrated in FIG. 9. Guilty subjects generally spent more time reading the neutral and exam items than the cash items. Follow-up tests indicated that guilty subjects generally spent more time reading the neutral items than the crime-related items, F(1,55)=21.96, partial η²=0.285, and more time reading the exam than the cash items, F(1,55)=104.74, partial η²=0.656. There were no significant differences between guilty and innocent subjects in responses to the three item types, although the difference between guilty and innocent subjects in time spent reading the neutral items was marginally significant, p=0.02.

The four-way interaction between guilt, motivation, sex, and question type was significant, F(2,192)=4.18, as was the five-way interaction between guilt, item difficulty, sex, question type, and repetition, F(8,768)=2.71.

### Reread Duration

The main effect of guilt was significant, F(1,96)=4.73. Guilty subjects generally did more rereading (M=0.016, SE=0.001) than did innocent subjects (M=0.013, SE=0.001). The effect of guilt was not moderated by motivation or item difficulty.

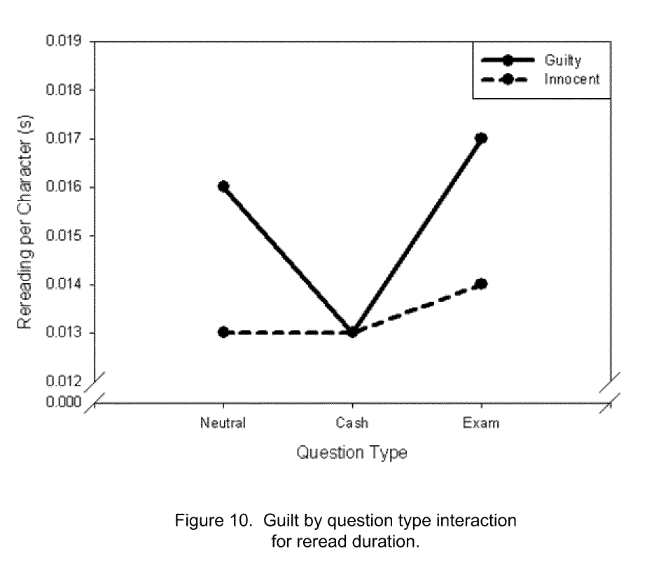

The guilt by question type interaction was significant, F(2,192)=15.17 and is illustrated in FIG. 10. Both groups did the same amount of rereading on cash items and did the most rereading on exam items. Follow-up tests indicated that guilty subjects generally did more rereading on exam items than cash items, F(1,55)=132.40, partial η²=0.707. The difference between neutral and crime-related items was marginally significant, F(1,55)=7.17, p=0.01, partial η²=0.115. Guilty subjects generally did more rereading on neutral items than crime-related items. Innocent subjects generally did more rereading on exam items than cash items, F(1,55)=37.21, partial η²=0.404. Follow-up tests also indicated guilty and innocent subjects differed in rereading on neutral items, p<0.01. The difference between guilty and innocent subjects on exam items was marginally significant, p=0.01. The guilt by question type interaction was not moderated by motivation or item difficulty.

The guilt by motivation by sex by repetition interaction was significant, F(4,384)=2.73.

### Pupil Diameter

PD was assessed by examining change from baseline. The first data point was subtracted from every subsequent data point in the response curve. A positive value indicated PD increased relative to baseline, and a negative value indicated PD decreased relative to baseline.

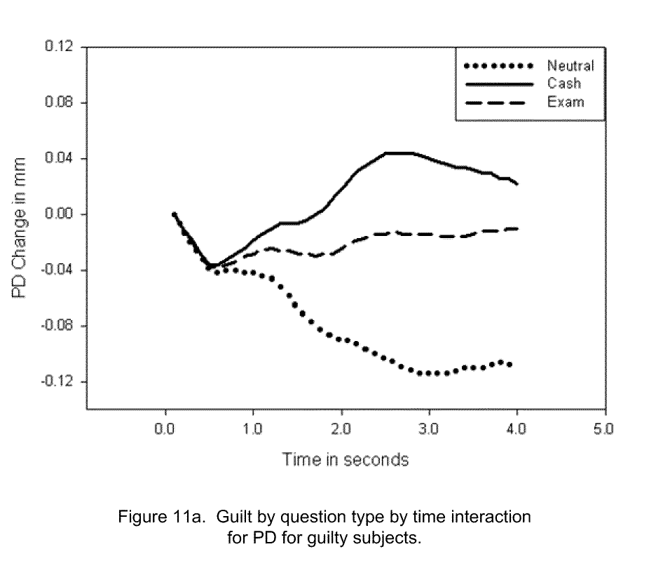

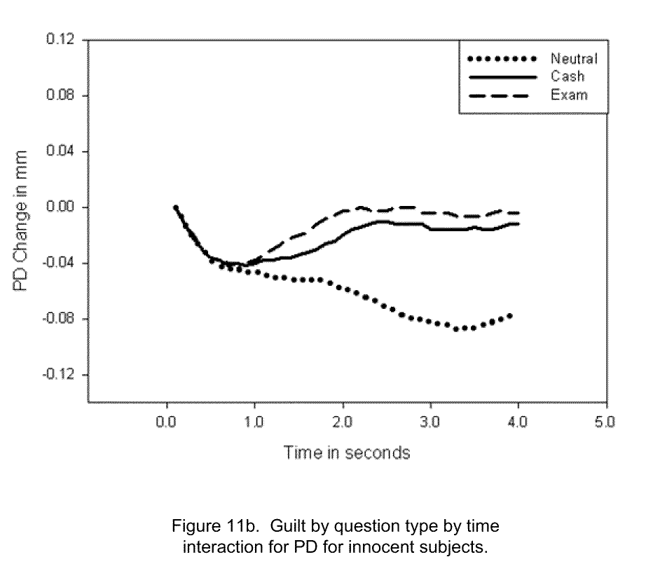

PD response curves for the guilt by question type by time interaction are illustrated in FIG. 11a and FIG. 11b for guilty and innocent subjects, respectively.

The guilt by question type interaction was significant, F(2,192)=17.89, as was the guilt by question type by time interaction, F(78,7488)=11.15. After an initial 500 ms decrease in PD, guilty subjects showed a greater increase in PD in response to crime items than to neutral items, F(1,55)=109.05, partial η²=0.665, and in response to cash items than to exam items, F(1,55)=20.75, partial η²=0.274. Innocent subjects generally showed a greater increase in PD to crime-related than to neutral items, F(1,55)=58.46, partial η²=0.515, with a slightly larger PD to exam than to cash items, F(1,55)=10.02, partial η²=0.154. Follow-up tests indicated that guilty and innocent subjects differed in PD responses to the cash items, p<0.01.

The guilt by repetition interaction also was significant, F(4,384)=3.36. The difference between guilty and innocent subjects varied significantly but not linearly across the five repetitions.

Two of the four-way interactions were statistically significant. The guilt by motivation by sex by time interaction was significant, F(39, 3744)=4.23, as was the guilt by question type by repetition by time interaction, F(312, 29952)=2.05.

PD responses to easy and difficult items within the mixed item difficulty condition also were examined. The main effect of difficulty was statistically significant, F(1,48)=40.83, partial η²=0.460. Subjects generally showed a greater change from baseline to the difficult items (M=−0.038, SE=0.006) than to the easy items (M=−0.021, SE=0.005).

### Item Blink Rate

The guilt by motivation by repetition interaction was statistically significant, F(4,384)=2.71. Follow-up analyses indicated the simple guilt by repetition interaction was marginally significant for the high motivation group, F(4,216)=3.69, p=0.01, partial η²=0.064, and not significant for the low motivation group, F(4,216)=0.66, p=0.56. In the high motivation condition, blink rate generally increased across repetitions for innocent subjects and decreased for guilty subjects.

The guilt by question type by sex interaction was significant, F(2,192)=3.91. Follow-up analyses indicated that the simple guilt by question type interaction was not significant for males or females at p<0.01.

The four-way interaction between guilt, motivation, item difficulty, and sex was marginally significant, F(1,96)=3.79, p=0.05, partial η²=0.038.

### Next Item Blink Rate

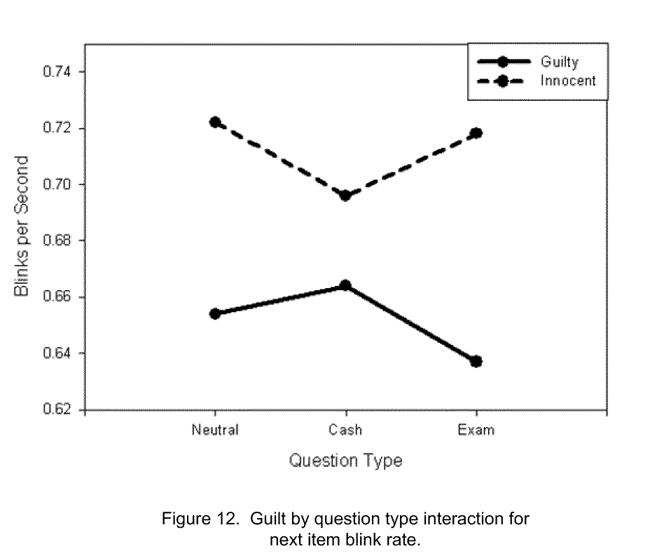

The guilt by question type interaction was statistically significant, F(2,192)=4.44, and is illustrated in FIG. 12. Guilty subjects generally showed the greatest increase in blink rate on items that followed a cash item. Innocent subjects generally showed the greatest increase in blink rate on items that followed neutral and exam items. Follow-up analyses comparing crime-related and neutral items and cash and exam items within the guilty and innocent groups were not significant at p<0.01, nor were there significant differences between the two groups for any of the three item types at p<0.01.

The guilt by motivation by repetition interaction was significant, F(4,384)=2.72. Follow-up analyses indicated the simple guilt by repetition interaction was significant for the high motivation group, F(4,216)=3.76, partial η²=0.065, but not the low motivation group, F(4,216)=0.53, p=0.64. In the high motivation condition, blink rate generally increased across repetitions for innocent subjects and decreased across repetitions for guilty subjects.

The four-way interaction between guilt, item difficulty, question type, and repetition also was statistically significant, F(8,768)=1.20. The four-way interaction between guilt, motivation, item difficulty, and sex was marginally significant, F(1,96)=3.90, p=0.051, partial η²=0.039.

### Example 3

### Classification of Guilty and Innocent Subjects

New dependent variables were created to develop discriminant functions. One dependent variable was the difference between the mean for crime-related items and the mean for neutral items. Another new dependent variable was created by computing the difference between the mean for cash items and the mean for exam items. The third new dependent variable was the mean for the neutral items. This procedure was used for all behavioral and oculomotor variables.

To assess the diagnostic validity of a derived outcome measure, it was correlated with a dichotomous variable that distinguished between guilty (coded 0) and innocent subjects (coded 1). To assess the reliability of the measure, responses were averaged within item types and within repetitions. This resulted in one mean for the neutral items, one mean for the cash items, and one mean for the exam items for each of the five repetitions. The difference between the crime items and the neutral items and the difference between the cash items and the exam items was computed for each repetition. Coefficient alpha then was computed to assess the internal consistency of the measures over repetitions.

The negative point-biserial correlations for RT, number of fixations, first pass duration, and reread duration for the neutral items indicate guilty subjects generally took longer to respond, made more fixations, and did more reading and rereading on neutral items as compared to innocent subjects. The correlations for the difference between the crime and neutral items and the difference between the cash and exam items for RT, number of fixations, first pass duration, and reread duration were generally positive. As compared to innocent subjects, guilty subjects generally took less time to respond, made fewer fixations, and did less reading and rereading on crime-related items than neutral items. Guilty subjects also generally took less time to respond, made fewer fixations, and did less reading and rereading of cash items than exam items. The point-biserial correlations and reliabilities for each measure are illustrated in FIG. 13 separately for the easy and mixed item difficulty conditions.

Eight variables then were selected for possible inclusion in the discriminant function: RTCrimeNeutral, RTCashExam, NFixCrimeNeutral, NFixCashExam, FirstPassCashExam, RereadCashExam, PDAreaCashExam, and NextItemBlinkRateCashExam. Seven of these variables were selected because they had point-biserial correlations of at least 0.30 in both the easy and mixed item difficulty groups. Although NextItemBlinkRateCashExam did not have a point-biserial correlation of at least 0.30 in both item difficulty groups, it was included because it was a variable of interest. PD area was selected to be consistent with prior studies.

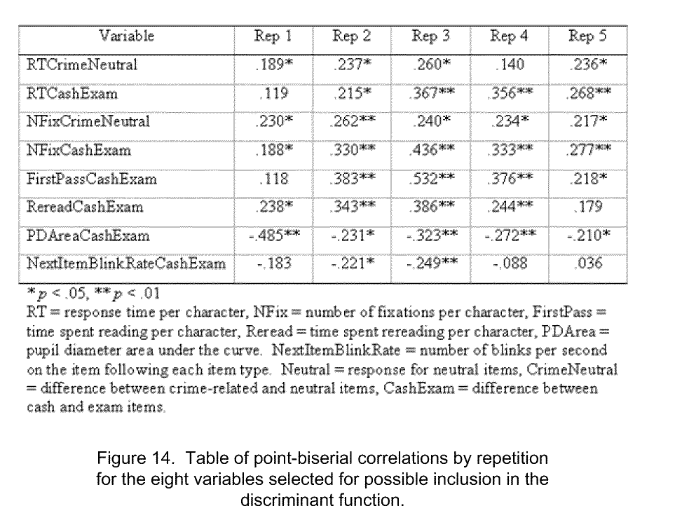

For each of the eight selected variables, the point-biserial correlation with guilt was computed for each repetition separately. Those correlations are illustrated in FIG. 14. The diagnostic validity appeared to vary across repetitions differently for the eight variables.

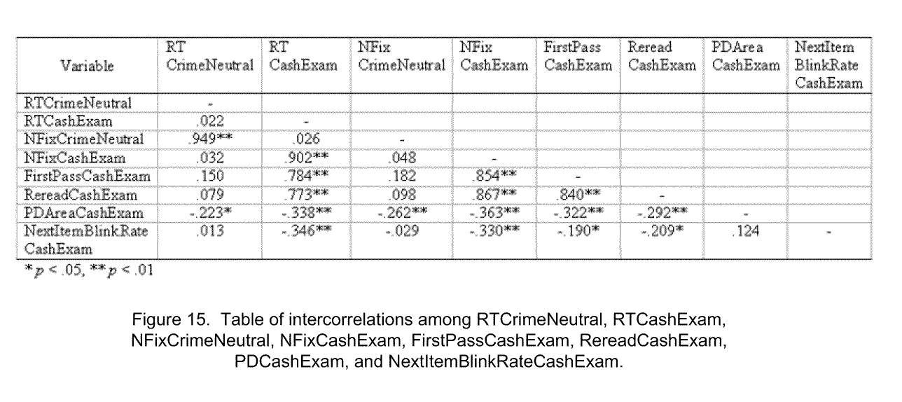

The intercorrelations among the eight variables are illustrated in FIG. 15. As expected, several potential predictor variables were highly intercorrelated.

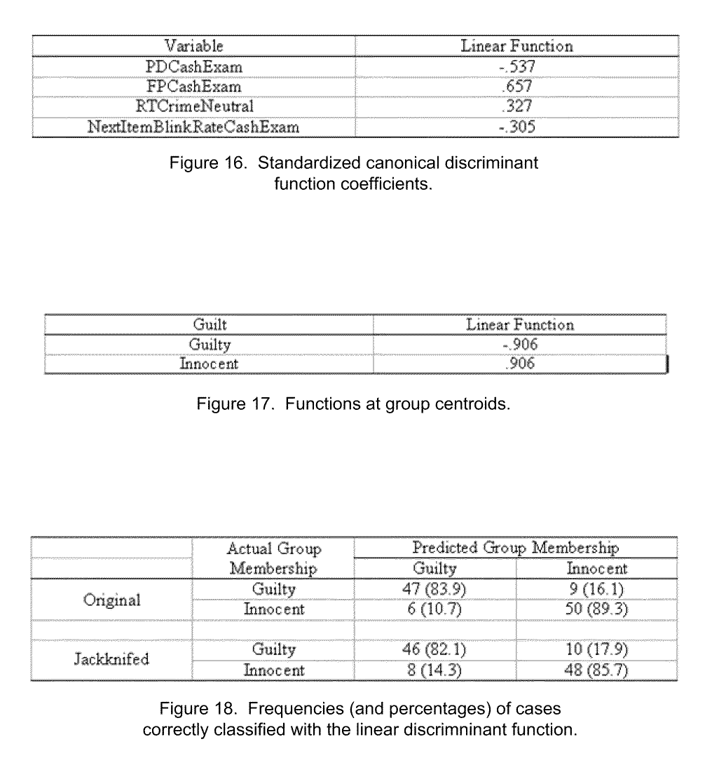

The eight variables were submitted to a stepwise multiple regression. Results indicated FirstPassCashExam, PDAreaCashExam, RTCrimeNeutral, and NextItemBlinkRateCashExam best predicted guilt. Coefficients for all four were statistically significant, ps<0.05. These four variables were used to create linear and quadratic discriminant functions and classification rates as previously described in W. W. Colley & P. R. Lohnes (1971), Multivariate data analysis, John Wiley & Sons, which is hereby incorporated by reference. The homogeneity of variance-covariance matrices assumption required for linear discriminant function analysis was not met, so quadratic analysis also was performed. Classification accuracy was poorer for the quadratic function. Only the simpler, linear solution is reported. The standardized canonical discriminant function coefficients and the functions at group centroids are illustrated in FIG. 16 and FIG. 17, respectively. Classification results and jackknifed classification results for the linear function is illustrated in FIG. 18. Jackknifed classification results were obtained with the leave-one-out method where each case was classified with coefficients computed from all other cases.

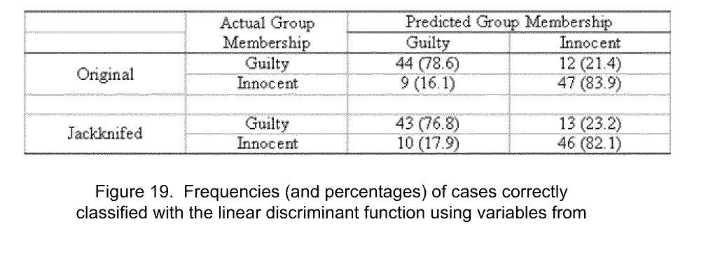

Four variables were used to create a linear discriminant function for this data. These four variables included PDCashCard, NFixCashCard, PDCrimeNeutral, and NFixNeutral. Classification rates for the linear function are illustrated in FIG. 19. These oculomotor measures were diagnostic of deception, and a weighted combination of four of those variables correctly classified 84% of guilty and 89% of innocent subjects. In other words, 84% were correctly classified as true positives (guilty classified guilty), 89% were correctly classified as true negatives (innocent classified innocent). As a result, 11% were misclassified as guilty—false positives (innocent classified guilty), and 16% were misclassified as innocent—false negative (guilty classified innocent).

### Example 4

### Effects of Self-Control and Achievement Motivation

Analyses were conducted to determine if self-control or achievement motivation moderate the relationship between guilt and RT and guilt and PD. RT for the difference between cash and exam items, RT for the neutral items, PD for the difference between cash and exam items, and PD for the neutral items were included as dependent variables. Guilt and self-control were centered on their respective means. Each dependent measure was regressed onto guilt, self-control, and their cross-product. The same logic was used to test if achievement motivation moderated the guilt by question type interaction.

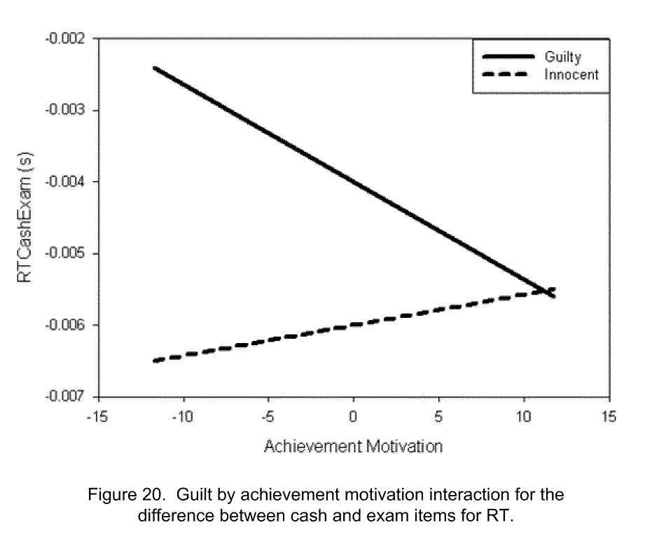

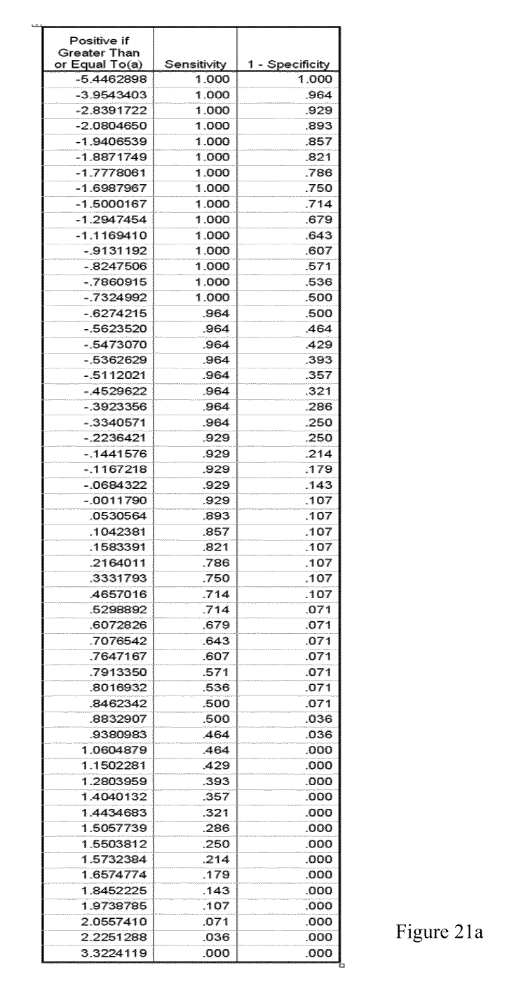

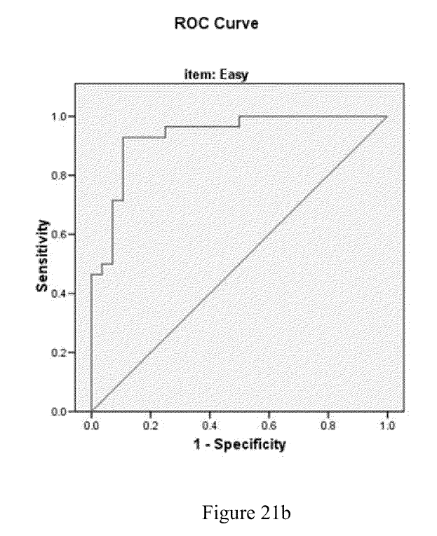

The cross-product for guilt and achievement motivation for the difference between RTs for cash and exam items was significant, p<0.05. This interaction is illustrated in FIG. 20. As compared to guilty subjects low in achievement motivation, innocent subjects low in achievement motivation generally took longer to respond to the exam items than to the cash items. There was little difference between guilty and innocent subjects high in achievement motivation.

### Example 5

### Interview Questions

Analyses of variance were performed on two of the interview questions subjects answered at the end of their session: anxiety at the beginning of the experiment and anxiety at the end of the experiment. Guilt, motivation, item difficulty, and sex were included as factors. Guilty subjects (M=3.491, SE=0.157) were more anxious than innocent subjects (M=2.652, SE=0.157) at the beginning of the experiment, F(1,96)=14.38, partial η²=0.130. Highly motivated subjects generally (M=3.304, SE=0.157) were more anxious than less motivated subjects generally (M=2.839, SE=0.157) at the beginning of the experiment, F(1,96)=4.40, partial η²=0.044. Females (M=3.348, SE=0.157) were generally more anxious than males (M=2.795, SE=0.157) at the beginning of the experiment, F(1,96)=6.25, partial η²=0.061.

There were no significant main or interaction effects for anxiety at the end of the experiment, although the guilt by motivation interaction was marginally significant, F(1,96)=3.81, p=0.054, partial η²=0.038. Guilty subjects in the high motivation condition were generally the most anxious at the end of the experiment (M=2.179, SE=0.188), followed by innocent subjects in the low motivation condition (M=1.679, SE=0.188), innocent subjects in the high motivation condition (M=1.571, SE=0.188), and guilty subjects in the low motivation condition (M=1.554, SE=0.188).

Chi-square analyses were conducted to test if responses to the question concerning speed versus accuracy when answering items and responses to the question asking which items were of most concern were related to guilt, motivation, item difficulty, or sex. None of the chi-squares were statistically significant, ps>0.16.

Subjects were asked how they would have approached the task if a different monetary bonus had been offered to pass the test, what strategies they used to try to pass the test, and how someone else could be taught to beat the test. When asked if they would have acted differently if offered a different monetary bonus ($1 for subjects in the high motivation condition and $30 for subjects in the low motivation condition), 58% of subjects stated they would have done nothing differently. For the subjects who stated they would have acted differently, most said they would have tried harder to beat the test to earn the larger bonus or not tried as hard to earn the smaller bonus. When asked if they used any strategies to try to convince the examiner of their innocence, 65% of subjects (44 guilty, 29 innocent) stated they had used strategies. Many stated they tried to be consistent in how they read and answered all items, answered as quickly and accurately as possible, took their time when answering neutral items, and remembered their answers from previous repetitions. Several of the guilty subjects stated that they tried to answer quickly when they were answering the cash items. When asked how they would teach someone else to beat the test, many subjects suggested that others read the items carefully, be consistent when reading and answering different item types, be calm and focused, and convince themselves of their innocence.

### Example 6

### The Impact of Next Blink Rate

To illustrate the impact of a single dependent measure on the predictive value of the discriminant function, the results above were recalculated excluding the single dependent measure NextItemBlinkRateCashExam. NextItemBlinkRateCashExam is particularly instructive of how the dependent measures collectively add to the overall discriminant function since it was one of the measures that did not have a point-biserial correlation of at least 0.30 in both item difficulty groups.

When NextItemBlinkRateCashExam was combined with NumberOfFixationsCashExam, PDAreaCashExam, PDAreaCrimeNeutral, and NumberOfFixationsNeutral, 85.7% of the truthful subjects were correctly identified and 78.6% of the deceptive subjects were correctly identified. When the NextItemBlinkRateCashExam variable was eliminated from the discriminate function, the accuracy on truthful subjects dropped from 85.7% to 83.9%, and the accuracy on deceptive subjects dropped from 78.6% to 75.0%. In other words, overall accuracy dropped by about 3%.

### Example 7

### Easy Mixed

To illustrate the impact of a single independent measure on the predictive value of the discriminant function, the results above were recalculated for easy and mixed (easy and difficult) items.

In the mixed items analysis, 22 of 28 deceptive subjects (78.6%) were correctly classified as deceptive; and 25 of 28 truthful subjects (89.3%) were correctly classified as truthful. In contrast, when the test was composed of only easy items, accuracy rates improved for deceptive and truthful subjects. Twenty-five of 28 deceptive subjects (89.3%) were correctly detected, and 26 of 28 truthful subjects (92.9%) were correctly classified as truthful Overall, the accuracy for subjects who received only easy items (91.1%) was 7% higher than the accuracy for the subjects who received mixed items (83.9%).

### Example 8

### Receiver Operator Curve

To illustrate the flexibility of the discriminant function, a receiver operator curve (ROC) was produced for the easy items discriminant function. This curve is a plot of the relationship between the sensitivity against 1-specificity for the particular discriminant function. In calculating the ROC in FIG. 21a, the smallest cutoff value is the minimum observed test value minus 1, and the largest cutoff value is the maximum observed test value plus 1. All the other cutoff values are the averages of two consecutive ordered observed test values.

As illustrated in FIG. 21b, the cutoff for the discriminant function can be set at any point on the curve depending on the desired outcome. For example, in certain situations it may be desirable to reduce the number of false positive (i.e., truthful subjects who are misclassified as deceptive) at the expense of true positives (i.e., deceptive subjects who are correctly classified as deceptive). Accordingly, the cutoff for the discriminant function may be set at a sensitivity of 46.4% and a specificity 0%. Conversely, it may be desirable to maximize the number of true positive (i.e., subjects who are correctly classified as deceptive) at the expense of false positives (i.e., truthful subjects who are misclassified classified as deceptive). In such a case, using the same data the cut off for the discriminant function may be set at a sensitivity of 100% and a specificity of 50%.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A computer-implemented method for assessing the veracity of a subject comprising:
presenting the subject with one or more sets of written test items relating to one or more issues of relevance;
presenting the subject with one or more sets of written test items relating to one or more neutral issues;
presenting the subject with one or more sets of written test items relating to one or more comparison issues;
monitoring, by a computing device, one or more dependent measures while the subject is reading and responding to said test items; and
analyzing, by the computing device, the variance of said dependent measures to assess veracity of said subject.

2. The method for assessing the veracity of a subject of claim 1, wherein the dependent measure is selected from the group consisting of response time, proportion wrong, number of fixations, first past duration, reread duration, pupil diameter, item blink rate and next item blink rate.

3. The method for assessing the veracity of a subject of claim 1, wherein a plurality of oculomotor dependent measures are analyzed to assess the veracity of said subject.

4. The method for assessing the veracity of a subject of claim 1, wherein the variance of a plurality of dependent measures as selected from Table 4 are analyzed to assess the veracity of said subject.

5. The method for assessing the veracity of a subject of claim 1, wherein the test items relating to issues of relevance, neutral issues and comparison issues comprises staggering the test items.

6. The method for assessing the veracity of a subject of claim 2, wherein a plurality of dependent measures are analyzed using a discriminant function and said discriminant function yields a sensitivity of at least 80%.

7. The method for assessing the veracity of a subject of claim 6, wherein a plurality of dependent measures are analyzed using a discriminant function and said discriminant function yields a specificity of at least 80%.

8. A computer-implemented method for assessing the veracity of a subject comprising:
presenting the subject with one or more sets of written test items relating to one or more issues of relevance;
presenting the subject with one or more sets of written test items relating to one or more neutral issues;
presenting the subject with one or more sets of written test items relating to one or more comparison issues;
wherein the subject is instructed to respond to the written test items as quickly and accurately as possible;
monitoring, by a computing device, one or more dependent measures while the subject is reading and responding to said test item; and
analyzing, by the computing device, the variance of said dependent measures together with the results from one or more independent variables to assess the veracity of said subject.

9. The method for assessing the veracity of a subject of claim 8, wherein the dependent measure is selected from the group consisting of response time, proportion wrong, number of fixations, first past duration, reread duration, pupil diameter, item blink rate and next item blink rate.

10. The method for assessing the veracity of a subject of claim 8, wherein a plurality of oculomotor dependent measures are analyzed to assess the veracity of said subject.

11. The method for assessing the veracity of a subject of claim 8, wherein the variance of a plurality of dependent measures as selected from Table 4 are analyzed to assess the veracity of said subject.

12. The method for assessing the veracity of a subject of claim 8, wherein the test items relating to issues of relevance, neutral issues and comparison issues comprises staggering the test items.

13. The method for assessing the veracity of a subject of claim 8, wherein a plurality of dependent measures are analyzed using a discriminant function and said discriminant function yields a sensitivity of at least 80%.

14. The method for assessing the veracity of a subject of claim 13, wherein a plurality of dependent measures are analyzed using a discriminant function and said discriminant function yields a specificity of at least 80%.

15. The method for assessing the veracity of a subject of claim 14, wherein a plurality of dependent measures and independent variables are analyzed using a discriminant function and said discriminant function yields a sensitivity of at least 80%.

16. The method for assessing the veracity of a subject of claim 15, wherein a plurality of dependent measures and independent variables are analyzed using a discriminant function and said discriminant function yields a specificity of at least 80%.

17. The method for assessing the veracity of a subject of claim 15, wherein only easy items are presented to the test subject.

18. The method for assessing the veracity of a subject of claim 17, wherein a plurality of dependent measures and independent variables are analyzed using a discriminant function and said discriminant function yields a accuracy of at least 80%.

19. A system for assessing the veracity of a subject comprising:
a display; and
a processor, wherein the processor is configured to:
present, on the display, the subject with one or more sets of written test items relating to one or more issues of relevance;
present, on the display, the subject with one or more sets of written test items relating to one or more neutral issues;
present, on the display, the subject with one or more sets of written test items relating to one or more comparison issues;
receive any responses from the subject related to the one or more issues of relevance, the one or more neutral issues, and the one or more comparison issues;
monitor one or more dependent measures while the subject is reading and responding to said test items; and
analyze the variance of said dependent measures to assess veracity of said subject.

20. The system of claim 19, wherein the one or more dependent measures are selected from the group consisting of response time, proportion wrong, number of fixations, first past duration, reread duration, pupil diameter, item blink rate and next item blink rate.
